SEQUENCE LISTING (3 sequences)

SEQ ID NO: 1 - protein, 1404 aa, Homo sapiens
MAWKTLPIYLLLLLSVFVIQQVSSQDLSSCAGRCGEGYSRDATCNCDYNCQHYMECCPDFKRVCTAELSCKGRCFESFERGRECDCDAQCKKYDKCCPDYESFCAEVHNPTSPPSSKKAPPPSGASQTIKSTTKRSPKPPNKKKTKKVIESEEITEEHSVSENQESSSSSSSSSSSSTIRKIKSSKNSAANRELQKKLKVKDNKKNRTKKKPTPKPPVVDEAGSGLDNGDFKVTTPDTSTTQHNKVSTSPKITTAKPINPRPSLPPNSDTSKETSLTVNKETTVETKETTTTNKQTSTDGKEKTTSAKETQSIEKTSAKDLAPTSKVLAKPTPKAETTTKGPALTTPKEPTPTTPKEPASTTPKEPTPTTIKSAPTTPKEPAPTTTKSAPTTPKEPAPTTTKEPAPTTPKEPAPTTTKEPAPTTTKSAPTTPKEPAPTTPKKPAPTTPKEPAPTTPKEPTPTTPKEPAPTTKEPAPTTPKEPAPTAPKKPAPTTPKEPAPTTPKEPAPTTTKEPSPTTPKEPAPTTTKSAPTTTKEPAPTTTKSAPTTPKEPSPTTTKEPAPTTPKEPAPTTPKKPAPTTPKEPAPTTPKEPAPTTTKKPAPTTPKEPAPTTPKETAPTTPKKLTPTTPEKLAPTTPEKPAPTTPEELAPTTPEEPTPTTPEEPAPTTPKAAAPNTPKEPAPTTPKEPAPTTPKEPAPTTPKETAPTTPKGTAPTTLKEPAPTTPKKPAPKELAPTTTKEPTSTTCDKPAPTTPKGTAPTTPKEPAPTTPKEPAPTTPKGTAPTTLKEPAPTTPKKPAPKELAPTTTKGPTSTTSDKPAPTTPKETAPTTPKEPAPTTPKKPAPTTPETPPPTTSEVSTPTTTKEPTTIHKSPDESTPELSAEPTPKALENSPKEPGVPTTKTPAATKPEMTTTAKDKTTERDLRTTPETTTAAPKMTKETATTTEKTTESKITATTTQVTSTTTQDTTPFKITTLKTTTLAPKVTTTKKTITTTEIMNKPEETAKPKDRATNSKATTPKPQKPTKAPKKPTSTKKPKTMPRVRKPKTTPTPRKMTSTMPELNPTSRIAEAMLQTTTRPNQTPNSKLVEVNPKSEDAGGAEGETPHMLLRPHVFMPEVTPDMDYLPRVPNQGIIINPMLSDETNICNGKPVDGLTTLRNGTLVAFRGHYFWMLSPFSPPSPARRITEVWGIPSPIDTVFTRCNCEGKTFFFKDSQYWRFTNDIKDAGYPKPIFKGFGGLTGQIVAALSTAKYKNWPESVYFFKRGGSIQQYIYKQEPVQKCPGRRPALNYPVYGETTQVRRRRFERAIGPSQTHTIRIQYSPARLAYQDKGVLHNEVKVSILWRGLPNVVTSAISLPNIRKPDGYDYYAFSKDQYYNIDVPSRTARAITTRSGQTLSKVWYNCP

SEQ ID NO: 2 - DNA, 5041 bp, Homo sapiens
gcggccgcgactattcggtacctgaaaacaacgatggcatggaaaacacttcccatttacctgttgttgctgctgtctgttttcgtgattcagcaagtttcatctcaagatttatcaagctgtgcagggagatgtggggaagggtattctagagatgccacctgcaactgtgattataactgtcaacactacatggagtgctgccctgatttcaagagagtctgcactgcggagctttcctgtaaaggccgctgctttgagtccttcgagagagggagggagtgtgactgcgacgcccaatgtaagaagtatgacaagtgctgtcccgattatgagagtttctgtgcagaagtgcataatcccacatcaccaccatcttcaaagaaagcacctccaccttcaggagcatctcaaaccatcaaatcaacaaccaaacgttcacccaaaccaccaaacaagaagaagactaagaaagttatagaatcagaggaaataacagaagaacattctgtttctgaaaatcaagagtcctcctcctcctcctcctcttcctcttcttcttcaacaatttggaaaatcaagtcttccaaaaattcagctgctaatagagaattacagaagaaactcaaagtaaaagataacaagaagaacagaactaaaaagaaacctacccccaaaccaccagttgtagatgaagctggaagtggattggacaatggtgacttcaaggtcacaactcctgacacgtctaccacccaacacaataaagtcagcacatctcccaagatcacaacagcaaaaccaataaatcccagacccagtcttccacctaattctgatacatctaaagagacgtctttgacagtgaataaagagacaacagttgaaactaaagaaactactacaacaaataaacagacttcaactgatggaaaagagaagactacttccgctaaagagacacaaagtatagagaaaacatctgctaaagatttagcacccacatctaaagtgctggctaaacctacacccaaagctgaaactacaaccaaaggccctgctctcaccactcccaaggagcccacgcccaccactcccaaggagcctgcatctaccacacccaaagagcccacacctaccaccatcaagtctgcacccaccacccccaaggagcctgcacccaccaccaccaagtctgcacccaccactcccaaggagcctgcacccaccaccaccaaggagcctgcacccaccactcccaaggagcctgcacccaccaccaccaaggagcctgcacccaccaccaccaagtctgcacccaccactcccaaggagcctgcacccaccacccccaagaagcctgccccaactacccccaaggagcctgcacccaccactcccaaggagcctacacccaccactcccaaggagcctgcacccaccaccaaggagcctgcacccaccactcccaaagagcctgcacccactgcccccaagaagcctgccccaactacccccaaggagcctgcacccaccactcccaaggagcctgcacccaccaccaccaaggagccttcacccaccactcccaaggagcctgcacccaccaccaccaagtctgcacccaccactaccaaggagcctgcacccaccactaccaagtctgcacccaccactcccaaggagccttcacccaccaccaccaaggagcctgcacccaccactcccaaggagcctgcacccaccacccccaagaagcctgccccaactacccccaaggagcctgcacccaccactcccaaggaacctgcacccaccaccaccaagaagcctgcacccaccgctcccaaagagcctgccccaactacccccaaggagactgcacccaccacccccaagaagctcacgcccaccacccccgagaagctcgcacccaccacccctgagaagcccgcacccaccacccctgaggagctcgcacccaccacccctgaggagcccacacccaccacccctgaggagcctgctcccaccactcccaaggcagcggctcccaacacccctaaggagcctgctccaactacccctaaggagcctgctccaactacccctaaggagcctgctccaactacccctaaggagactgctccaactacccctaaagggactgctccaactaccctcaaggaacctgcacccactactcccaagaagcctgcccccaaggagcttgcacccaccaccaccaaggagcccacatccaccacctctgacaagcccgctccaactacccctaaggggactgctccaactacccctaaggagcctgctccaactacccctaaggagcctgctccaactacccctaaggggactgctccaactaccctcaaggaacctgcacccactactcccaagaagcctgcccccaaggagcttgcacccaccaccaccaaggggcccacatccaccacctctgacaagcctgctccaactacacctaaggagactgctccaactacccccaaggagcctgcacccactacccccaagaagcctgctccaactactcctgagacacctcctccaaccacttcagaggtctctactccaactaccaccaaggagcctaccactatccacaaaagccctgatgaatcaactcctgagctttctgcagaacccacaccaaaagctcttgaaaacagtcccaaggaacctggtgtacctacaactaagactcctgcagcgactaaacctgaaatgactacaacagctaaagacaagacaacagaaagagacttacgtactacacctgaaactacaactgctgcacctaagatgacaaaagagacagcaactacaacagaaaaaactaccgaatccaaaataacagctacaaccacacaagtaacatctaccacaactcaagataccacaccattcaaaattactactcttaaaacaactactcttgcacccaaagtaactacaacaaaaaagacaattactaccactgagattatgaacaaacctgaagaaacagctaaaccaaaagacagagctactaattctaaagcgacaactcctaaacctcaaaagccaaccaaagcacccaaaaaacccacttctaccaaaaagccaaaaacaatgcctagagtgagaaaaccaaagacgacaccaactccccgcaagatgacatcaacaatgccagaattgaaccctacctcaagaatagcagaagccatgctccaaaccaccaccagacctaaccaaactccaaactccaaactagttgaagtaaatccaaagagtgaagatgcaggtggtgctgaaggagaaacacctcatatgcttctcaggccccatgtgttcatgcctgaagttactcccgacatggattacttaccgagagtacccaatcaaggcattatcatcaatcccatgctttccgatgagaccaatatatgcaatggtaagccagtagatggactgactactttgcgcaatgggacattagttgcattccgaggtcattatttctggatgctaagtccattcagtccaccatctccagctcgcagaattactgaagtttggggtattccttcccccattgatactgtttttactaggtgcaactgtgaaggaaaaactttcttctttaaggattctcagtactggcgttttaccaatgatataaaagatgcagggtaccccaaaccaattttcaaaggatttggaggactaactggacaaatagtggcagcgctttcaacagctaaatataagaactggcctgaatctgtgtattttttcaagagaggtggcagcattcagcagtatatttataaacaggaacctgtacagaagtgccctggaagaaggcctgctctaaattatccagtgtatggagaaatgacacaggttaggagacgtcgctttgaacgtgctataggaccttctcaaacacacaccatcagaattcaatattcacctgccagactggcttatcaagacaaaggtgtccttcataatgaagttaaagtgagtatactgtggagaggacttccaaatgtggttacctcagctatatcactgcccaacatcagaaaacctgacggctatgattactatgccttttctaaagatcaatactataacattgatgtgcctagtagaacagcaagagcaattactactcgttctgggcagaccttatccaaagtctggtacaactgtccttagactgatgagcaaaggaggagtcaactaatgaagaaatgaataataaattttgacactgaaaaacattttattaataaagaatattgacatgagtataccagtttatatataaaaatgtttttaaacttgacaatcattacactaaaacagatttgataatcttattcacagttgttattgtttacagaccatttaattaatatttcctctgtttattcctcctctccctcccattgcatggctcacacctgtaaaagaaaaaagaatcaaattgaatatatcttttaagaattcaaaactagtgtattcacttaccctagttcattataaaaaatatctaggcattgtggatataaaactgttgggtattctacaacttcaatggaaattattacaagcagattaatccctctttttgtgacacaagtacaatctaaaagttatattggaaaacatggaaatattaaaattttacacttttactagctaaaacataatcacaaagctttatcgtgttgtataaaaaaattaacaatataatggcaataggtagagatacaacaaatgaatataacactataacacttcatattttccaaatcttaatttggatttaaggaagaaatcaataaatataaaatataagcacatatttattatatatctaaggtatacaaatctgtctacatgaagtttacagattggtaaatatcacctgctcaacatgtaattatttaataaaactttggaacattaaaaaaataaattggaggcttaaaaaaaaaaaaaaaaa

SEQ ID NO: 3 - protein, 7 aa, Homo sapiens
KEPAPTT

(19) United States
(12) Patent Application Publication
(10) Pub. No.: US 2021/0338774 A1
(43) Pub. Date: Nov. 4, 2021

(54) PRG-4 FOR TREATING GOUT AND ITS SYMPTOMS

(71) Applicant: Lubris LLC, Naples, FL (US)

(72) Inventors: Gregory D. Jay, Norfolk, MA (US); Khaled Elsaid, Irvine, CA (US)

(21) Appl. No.: 17/318,198

(22) Filed: May 12, 2021

Related U.S. Application Data
(63) Continuation of application No. 15/808,632, filed on Nov. 9, 2017, now abandoned.
Application No. 15/808,632 is a continuation-in-part of application No. 15/546,192, filed on Jul. 25, 2017, now Pat. No. 10,967,048.
(60) Provisional application No. 62/420,975, filed on Nov. 11, 2016.
(60) Provisional application No. 62/273,059, filed on Dec. 30, 2015.
(60) Provisional application No. 62/107,799, filed on Jan. 26, 2015.

(51) Int. Cl. A61K 38/17; A61P 19/02; A61K 9/00

(52) U.S. Cl. CPC A61K 38/1709; A61K 9/0019; A61P 19/02

(57) ABSTRACT

Disclosed are methods of treating gout in a subject and methods of reducing joint pain in a subject with gout or pseudogout, comprising administering to the subject a composition comprising PRG4 or a biologically active fragment thereof.

### CROSS REFERENCE TO RELATED APPLICATIONS

This application is a continuation of U.S. patent application Ser. No. 15/808,632 filed Nov. 9, 2017, which claims priority to and the benefit of U.S. Provisional Patent Application No. 62/420,975 filed Nov. 11, 2016, and is a continuation-in-part of U.S. patent application Ser. No. 15/546,192 filed Jul. 25, 2017, which is a U.S. national stage application filed under 35 U.S.C. § 371 of International Patent Application No. PCT/US2016/014952 filed Jan. 26, 2016, which claims priority to and the benefit of U.S. Provisional Patent Application No. 62/273,059 filed Dec. 30, 2015, and U.S. Provisional Patent Application No. 62/107,799 filed Jan. 26, 2015. The contents of each of the applications to which priority is claimed are incorporated by reference herein in their entireties.

### FIELD OF THE INVENTION

The field of the invention is treating gout and the symptoms of gout.

### BACKGROUND OF THE INVENTION

Gout is a common inflammatory arthritis characterized by elevated serum uric acid levels and deposition of monosodium urate monohydrate (MSU) crystals in synovial joints and periarticular tissues (Pascual E et al., Nature Reviews Rheumatology 2015; 11:725-730; Bitik B et al., Eur J Rheumatol 2014; 1(2):72-77). Gout is characterized by painful episodes of acute monoarthritis, more likely to happen in the first metatarsophalangeal and knee joints, interspersed by asymptomatic periods (Bitik B et al., Eur J Rheumatol 2014; 1(2):72-77; Stewart S et al., BMC Musculoskeletal Disord 2016; 17:69). Complications of gout include the development of tophi and articular surface damage. The prevalence and incidence of gout is rising globally with an estimated prevalence of 1-2% in the adult population in Europe and up to 4% in the population in the U.S (Zhu Y et al., Arthritis Rheum 2011; 63:3136-3141; Smith E et al., Ann Rheum Dis 2014; 73(8):1470-6; Kuo C F et al., Nat Rev Rheumatol 2015; 11(11):649-62).

MSU crystals trigger inflammation in the joint by a mechanism that involves phagocytosis by resident macrophages resulting in activation of the inflammasome and induction of the expression and secretion of pro-inflammatory cytokines, e.g. interleukin-1 beta (IL-1β), tumor necrosis factor alpha (TNF-α) and interlukin-6 (IL-6) (Busso N et al., Arthritis Res Ther 2010; 12(2):206; di Giovine F S et al., J Clin Invest. 1991; 87:1375-1381; Martin W J et al., Arthritis Rheum 2009; 60:281-289; Chen C J et al., J Clin Invest 2006; 116:2262-2271). Macrophage activation by MSU crystals results in induction of the gene expression and secretion of chemokines, e.g. interleukin-8 (IL-8), monocyte chemoattractant protein-1 (MCP-1) and GROα resulting in neutrophil and monocyte chemotaxis and joint influx that sustains the acute inflammatory response (Nishimura A et al., J Leukoc Biol 1997; 62:444-449; Pope R M et al., Arthritis Rheum 2007; 56(10):3183-3188; Pessler F et al., Arthritis Res Ther 2008; 10:R64;). The mechanism of MSU phagocytosis by macrophages is not clearly defined. Toll-like receptors 2 and 4 (TLR2 and TLR4) may mediate MSU uptake by macrophages. Bone marrow derived macrophages from TLR2 and TLR4 knockout mice show decreased MSU uptake and macrophage activation compared to their congenic wild type (Bryan R L et al., Arthritis Rheum 2005; 52(9):2936-2946).

Current treatments for gout and associated joint pain include traditional pain relievers such as non-steroidal anti-inflammatory drugs (NSAIDs) such as ibuprofen, naproxen sodium, indomethacin, or celecoxib. However, these drugs carry the risk of stomach pain, bleeding, and ulcers. Colchicine is another type of pain reliever that is used for relief of pain associated with gout. However, it has serious side effects such as nausea, vomiting, and diarrhea; such side effects often offset the benefits of the drug's effectiveness. Corticosteroids are another common treatment for gout related pain; however, the side effects include increased blood sugar levels, elevated blood pressure, and even mood changes. Drugs designed to block uric acid production (xanthine oxidase inhibitors such as allopurinol, febuxostat) or improve uric acid removal (e.g., probenecid) are also used to treat gout; however, their side effects include stomach pain, kidney stones, nausea, and reduced liver function. Accordingly, new and novel treatments for gout and the symptoms of gout are needed, especially those having limited to no side effects.

Lubricin/Proteoglycan-4 (PRG4) is a mucinous glycoprotein secreted by synovial fibroblasts and superficial zone articular chondrocytes (Jay G D et al., J Rheumatol 2000; 27(3):594-600; Jay G D et al., J Orthop Res 2001; 19(4):677-87; Flannery C R et al., Biochem Biophys Res Commun 1999; 254(3):535-541). PRG4 is a major constituent of synovial fluid (SF) and a biological role for PRG4 has been described. PRG4 may be useful in the treatment of gout and its symptoms including joint pain and allodynia.

### SUMMARY OF THE INVENTION

It has now been discovered that PRG4 is useful in the treatment of gout and its symptoms including joint pain and allodynia.

The invention is directed to a method of reducing joint pain in a subject with gout by administering to the subject a composition comprising PRG4 or a homolog or biologically active fragment thereof. The invention is directed to a method of reducing joint pain in a subject with pseudogout by administering to the subject a composition comprising PRG4 or a homolog or biologically active fragment thereof.

The invention is also directed to a method of treating gout in a subject by administering to the subject a composition comprising PRG4 or a homolog or a biologically active fragment thereof. The invention is also directed to a method of treating pseudogout in a subject by administering to the subject a composition comprising PRG4 or a homolog or a biologically active fragment thereof.

The invention is also directed to a method of decreasing phagocytosis of monosodium urate monohydrate (MSU) crystals by a macrophage in a patient suffering from gout. The method involves administering to the subject a composition comprising PRG4 or a homologue or biologically active fragment thereof.

The invention is also directed to a method of treating gout in a patient by reducing inflammation associated with gout. The method involves administering to the patient a composition comprising PRG4 or a homologue or biologically active fragment thereof.

The invention is also directed to a method of treating gout in a patient by reducing inflammation associated with pseudogout. The method involves administering to the patient a composition comprising PRG4 or a homologue or biologically active fragment thereof.

In some embodiments, the PRG4 is recombinant human PRG4.

In some embodiments, the PRG4 is administered to the location of MSU crystals in the patient, for example, to a joint affected by gout by intra-articular injection. The joint may be, for example, a knee, ankle, elbow, shoulder, finger, thumb, wrist, or toe joint. In some embodiments, the PRG4 is administered to the subject by injection into area of the patient's body affected by gout. The affected area may be, for example, the heel or instep of the patient's foot.

In some embodiments, the PRG4 is administered to the location of calcium pyrophosphate dehydrate crystals in a patient experiencing pseudogout, for example, to the affected joint.

In some embodiments, the PRG4 is administered systemically, e.g., intravenously, to the patient affected by gout or pseudogout.

In some embodiments, the administered composition further comprises a pharmaceutical carrier in addition to PRG4.

In some embodiments, the PRG4 is administered in an amount insufficient to provide boundary lubrication. The PRG4 may be administered in an amount sufficient to treat joint pain or allodynia. The PRG4 may be administered in an amount sufficient to treat gout. The PRG4 may be administered in an amount sufficient to treat pseudogout. The PRG4 may be administered in an amount sufficient to decrease macrophage phagocytosis of MSU crystals. The PRG4 may also be administered in an amount sufficient to reduce inflammation associated with gout or pseudogout.

In some embodiments, the PRG4 administered is in the range of 0.1 μg/kg to 4000 μg/kg, or 0.1 μg/kg to 1000 μg/kg, or 0.1 μg/kg to 100 μg/kg, or 0.1 to 50 μg/kg. In some embodiments, the PRG4 administered is in the range of 0.1 μg/mL to 30 mg/mL, or 1 μg/mL to 10 mg/mL, or 10 μg/mL to 1 mg/mL. In some embodiments, the PRG4 administered is in the range of 2 mg to 10 mg, 2 mg to 5 mg, 5 mg to 10 mg or greater than 10 mg. In other embodiments, the PRG4 is administered sufficient to achieve a concentration of PRG4 in a synovial fluid of a joint of the subject of at least 200 μg/ml, at least 300 μg/ml, at least 400 μg/ml, at least 500 μg/ml, or at least 1000 μg/ml. The PRG4 may be administered weekly, biweekly, or monthly or quarterly.

In some embodiments, the subject is a mammal. For example, the subject is a human, horse, sheep, pig, dog, or cat.

In some embodiments, PRG4 or the biologically active fragment of PRG4 has at least 80%, at least 85%, at least 90%, at least 95%, at least 96%, at least 97%, at least 98%, or at least 99% sequence identity with SEQ ID NO:1.

### BRIEF DESCRIPTION OF THE FIGURES

FIGS. 1A-E show the phagocytosis of monosodium urate monohydrate (MSU) crystals by THP-1 macrophages and the impact of anti-toll-like receptor 2 (TLR2) antibody or recombinant human proteoglycan-4 (rhPRG4) treatments. FIG. 1A shows representative flow cytometry scatter plot of untreated THP-1 macrophages, MSU-treated and MSU+anti-TLR2 antibody-treated macrophages following incubation for 6 hours. MSU crystals were phagocytized by THP-1 macrophages as evidenced by an increase in cell population side scatter (SSc), and anti-TLR2 antibody (20 μg/mL) treatment reduced cell population SSc. FIG. 1B shows anti-TLR2 antibody (20 μg/mL) treatment inhibited MSU phagocytosis by THP-1 macrophages. Data represents the mean±S.D. of three independent experiments. *p<0.001; **p<0.01. FIG. 1C shows association of rhPRG4 with THP-1 macrophages. Rhodamine-labeled rhPRG4 (20 μg/mL) associated with THP-1 macrophages following incubation for 6 hours. A threshold was set at red fluorescence intensity=10. Cell-associated fluorescence higher than 10 was considered positive. FIG. 1D representative flow cytometry scatter plot of untreated THP-1 macrophages, MSU-treated and MSU+rhPRG4 treated macrophages following incubation for 6 hours. MSU crystals were phagocytized by THP-1 macrophages as evidenced by an increase in cell population side scatter (SSc), and rhPRG4 (200 μg/mL) treatment reduced cell population SSc. FIG. 1E rhPRG4 (200 μg/mL) treatment inhibited MSU phagocytosis by THP-1 macrophages. Data represents the mean±S.D. of three independent experiments. *p<0.001; **p<0.01.

FIGS. 2A-D show the impact of recombinant human proteoglycan-4 (rhPRG4) treatment on monosodium urate monohydrate (MSU) crystal-induced gene expression of pro-inflammatory cytokines and chemokines in THP-1 macrophages. Data is presented as fold induction of pro-inflammatory cytokines and chemokines gene expression compared to control untreated THP-1 macrophages. Data represents the mean±S.D. of 3 independent experiments. *p<0.001; **p<0.01. FIG. 2A rhPRG4 (100 and 200 μg/mL) treatment reduced interleukin-1 beta (IL-1β) gene expression in MSU-stimulated THP-1 macrophages. FIG. 2B shows rhPRG4 (25, 50, 100 and 200 μg/mL) treatment reduced tumor necrosis factor alpha (TNF-α) gene expression in MSU-stimulated THP-1 macrophages. FIG. 2C rhPRG4 (50, 100 and 200 μg/mL) treatment reduced monocyte chemoattractant protein-1 (MCP-1) gene expression in MSU-stimulated THP-1 macrophages. FIG. 2D rhPRG4 (50, 100 and 200 μg/mL) treatment reduced interleukin-8 (IL-8) gene expression in MSU-stimulated THP-1 macrophages.

FIGS. 3A-3D show the impact of recombinant human proteoglycan-4 (rhPRG4) treatment on monosodium urate monohydrate (MSU) crystal-induced pro-inflammatory cytokines and chemokines production by THP-1 macrophages. Media concentrations of pro-inflammatory cytokines and chemokines of MSU-stimulated macrophages in the presence or absence of rhPRG4 are presented as percent of media concentrations from untreated control THP-1 macrophages. Data represents the mean±S.D. of 3 independent experiments with duplicate wells per group. *p<0.001; **p<0.01. FIG. 3A shows rhPRG4 (200 μg/mL) treatment reduced interleukin-1 beta (IL-1β) production by MSU-stimulated THP-1 macrophages. FIG. 3B shows rhPRG4 (100 and 200 μg/mL) treatment reduced tumor necrosis factor alpha (TNF-α) production by MSU-stimulated THP-1 macrophages. FIG. 3C shows rhPRG4 (200 μg/mL) treatment reduced monocyte chemoattractant protein-1 (MCP-1) production by MSU-stimulated THP-1 macrophages. FIG. 3D shows rhPRG4 (100 and 200 μg/mL) treatment reduced interleukin-8 (IL-8) production by MSU-stimulated THP-1 macrophages.

FIGS. 4A-C show phagocytosis of monosodium urate monohydrate (MSU) crystals by peritoneal murine macrophages from Prg4^{+/+} and Prg4^{-/-} mice and the impact of anti-toll-like receptor 2 (TLR2) antibody or recombinant human proteoglycan-4 (rhPRG4) treatments. FIG. 4A shows representative images of DAPI-stained peritoneal macrophages from Prg4^{+/+} and Prg4^{-/-} mice following incubation with MSU crystals for 6 hours in the presence or absence of anti-TLR2 antibody or rhPRG4. Arrows point to MSU crystals localized intracellularly. Anti-TLR2 antibody or rhPRG4 treatments reduced MSU phagocytosis by peritoneal macrophages. FIG. 4B shows Anti-TLR2 antibody (20 μg/mL) and rhPRG4 (200 μg/mL) treatments reduced MSU phagocytosis by peritoneal macrophages from Prg4^{+/+} and Prg4^{-/-} mice following incubation for 6 hours. Data represents the mean±S.D. of 5 independent experiments. *p<0.001; **p<0.01; ***p<0.05. FIG. 4C shows rhPRG4 (200 μg/mL) treatment reduced interleukin-1 beta (IL-1β) production by MSU-stimulated peritoneal murine macrophages from Prg4^{+/+} and Prg4^{-/-} mice following incubation for 24 hours. Data represents the mean±S.E.M of 5 independent experiments. *p<0.001.

FIG. 5 shows fold changes of IL-1RA RNA expression in Prg4^{-/-} compared with Prg4^{+/+} peritoneal murine macrophages after the cells were treated with MSU or with MSU and rhPRG4.

FIGS. 6A-B shows the impact of recombinant human proteoglycan-4 (rhPRG4) treatment on differential weight bearing (Right hind limb-Left hind limb; R-L) and paw withdrawal threshold (PWT) following intra-articular administration of monosodium urate monohydrate (MSU) crystals (50 μL; 2.5 mg/mL) in the right knee joint of male Lewis rats followed by intra-articular treatments with rhPRG4 (1 mg/mL; 50 μL) or PBS (50 μL) at 1 hour following MSU administration. Differential weight bearing was measured at 3 hours (n=14 in each group), 6 hours (n=14 in each group) and 24 hours (n=8 in each group) following MSU administration. PWT measurements were performed at 6 hours (n=14 in each group) and 24 hours (n=8 in each group) following MSU administration. Data is presented as mean±S.D. *p<0.001; **p<0.01; ***p<0.05. FIG. 6A rhPRG4 treatment decreased differential weight bearing at 6 hours compared to PBS. FIG. 6B rhPRG4 treatment increased PWT at 6 hours compared to PBS.

FIG. 7 is the amino acid sequence of full length (non-truncated) human PRG4 (SEQ ID NO:1: 1404 residues). Residues 1-24 (shown in bold) represent the signal sequence and residues 25-1404 represent the mature sequence of human PRG4. The glycoprotein does not require the lead sequence in its active form.

FIGS. 8A-C together provide the complete nucleic acid sequence for the PRG4 gene (SEQ ID NO:2) which encodes the full length 1404 AA human PRG4 protein. FIG. 8A provides residues 1-2080 of the nucleic acid sequence of SEQ ID NO:2. FIG. 8B provides residues 2081-4160 of the nucleic acid sequence of SEQ ID NO:2. FIG. 8C provides residues 4161-5041 of the nucleic acid sequence of SEQ ID NO:2. SEQ ID NO:2 has a total length of 5041 residues, represents the DNA sequence from Homo sapiens, and has GenBank Accession No. U70136.1.

FIGS. 9A-C are bar graphs showing the levels of IL-1β, IL-8, and MCP-1 in pg/ml differentiated human THP-1 macrophages treated with MSU, MSU and rhPRG4, MSU and bovine submaxillary mucin (BSM) (positive control), rhPRG4, or BSM. (*p<0.001; **p<0.01)

### DETAILED DESCRIPTION OF THE INVENTION

The invention disclosed herein is based on the discovery that PRG4, also known as lubricin, has the ability to treat gout and symptoms related to gout such as allodynia or joint pain in a patient. The invention is also based on the discovery that administration of PRG4 can decrease macrophage phagocytosis of MSU crystals and reduce inflammation and joint pain associated with gout.

While pseudogout is a disorder that has a different pathogenesis that gout, being caused by deposition of calcium pyrophosphate dehydrate crystals in connective tissues, such as a joint, the invention contemplates that PRG4 can be used to treat pseudogout and associated joint inflammation and other symptoms. It is proposed that the mechanism of action of PRG4 in treating pseudogout is, in principle, similar to the mechanism of action of PRG4 in treating gout as described herein.

Lubricin/Proteoglycan-4 (PRG4) is a mucinous glycoprotein secreted by synovial fibroblasts and superficial zone articular chondrocytes (Jay G D et al., J Rheumatol 2000; 27(3):594-600; Jay G D et al., J Orthop Res 2001; 19(4):677-87; Flannery C R et al., Biochem Biophys Res Commun 1999; 254(3):535-541). PRG4 is a major constituent of synovial fluid (SF) and a biological role for PRG4 has been described. The recombinant form of PRG4 exhibits a CD-44 mediated anti-inflammatory role characterized by its ability to inhibit IL-1β and TNF-α induced nuclear factor kappa b (NFκB) nuclear translocation in synoviocytes from patients with rheumatoid arthritis (Al-Sharif A et al., Arthritis Rheumatol 2015; 67(6):1503-1513.). Recombinant human PRG4 (rhPRG4) also binds to, and regulates agonist-induced activation of TLR2 and TLR4 (Iqbal S M et al., Sci. Rep. 2016; 6:18910; Alquraini A et al., Arthritis Res Ther 2015; 17:353). PRG4 in SF aspirates from patients with osteoarthritis (OA) inhibits the activation of TLR2 by TLR2 ligands in OA SF (Alquraini A et al., Arthritis Res Ther 2015; 17:353). As shown by the data presented herein, rhPRG4 inhibits MSU phagocytosis by macrophages, similar to TLR2 neutralization, resulting in a significant reduction in IL-β, TNF-α, IL-8 and MCP-1 expression and production and an anti-nociceptive effect in an acute gout model in vivo.

In acute gout, MSU-crystals liberated from tissue deposits are phagocytosed by macrophages and promote an inflammatory cascade that involves complement activation and release of multiple inflammatory cytokines, which culminate in an acute neutrophilic inflammation (Chen C J et al., J Clin Invest 2006, 116(8):2262-2271; Liu-Bryan R et al., Arthritis Rheum 2005, 52(9):2936-2946) that can recruit peripheral monocytes to the joint in an IL-1β dependent manner (autoinduction loop). IL-1β has a more central role than tumor necrosis factor in experimental urate-crystal-induced inflammation (Edwards N L et al., Rheum Dis Clin North Am 2014, 40(2):375-387). At the cellular level, a fundamental mechanism that promotes MSU-crystal-induced inflammation is innate immune engagement of the crystals by plasma membrane receptors, including Toll-like receptors (TLRs) 2 and 4, on mononuclear phagocytes (Samsom M L et al., Experimental eye research 2014, 127:14-19).

rhPRG4 also plays an anti-inflammatory role through its ability to prevent NF-kB translocation in fibroblasts stimulated with TNFα through CD44 inhibition and concentration dependent binding to TLR2 and TLR4 (Al-Sharif A et al., Arthritis & rheumatology 2015, 67(6):1503-1513; Iqbal S M et al., Scientific reports 2016, 6:18910; Alquraini A et al., Arthritis Res Ther 2015, 17:353). As demonstrated herein, human THP-1 monocytes treated with PMA also show MSU uptake that can be inhibited by rhPRG4. Further, as demonstrated in this application, rhPRG4 may engage the NLRP3 inflammasome through its internalization where it may block the cleavage of pro-IL-1β and thus secretion of mature IL-1β. Without wishing to be bound by theory, this may explain why rhPRG4 may be effective as an intraarticular therapy in acute gouty arthritis. However, as demonstrated herein, a proposed mechanism of action of PRG4 may occur upstream of IL-1β expression in re-establishing the expression of IL-1ra in PBMCs that is TLR dependently suppressed by elevated serum urate (Crisan T O et al., Ann Rheum Dis 2016, 75(4):755-762). Less IL-1ra results in unapposed effects of IL-1β on IL1R and promulgation of the IL-1β autoinduction loop.

In one embodiment, the invention provides a method for reducing joint pain or allodynia in a patient with gout. The method involves administering to the subject a composition comprising PRG4 or a biologically active homolog or variant thereof. In another embodiment, the invention also provides a method for treating gout. The method involves administering to the subject a composition comprising PRG4 or a biologically active homolog or variant thereof. In a further embodiment, the invention also provides a method for reducing macrophage phagocytosis of MSU crystals. The method involves administering to the subject a composition comprising PRG4 or a biologically active homolog or variant thereof. In yet another, embodiment, the invention also provides a method for reducing inflammation associated with gout. The method involves administering to the subject a composition comprising PRG4 or a biologically active homolog or variant thereof. In yet another embodiment, the invention provides a method for treating pseudogout and inflammation associated with pseudogout, such as joint inflammation or synovitis, as well as for treating joint pain or allodynia associate with pseudogout. The method involves administering a composition comprising PRG4 or a biologically active homolog or variant thereof to the patient.

In a further embodiment, PRG4 is administered to a patient experiencing a gouty flare up. In yet another embodiment, PRG4 is administered to a patient with gout to prevent a flare up from occurring. Administration is either intra-articular or intravenous.

### PRG4 Protein

PRG4, also referred to as lubricin, is a lubricating polypeptide, which in humans is expressed from the megakaryocyte stimulating factor (MSF) gene, also known as PRG4 (see NCBI Accession Number AK131434-U70136). Lubricin is a ubiquitous, endogenous glycoprotein that coats the articulating surfaces of the body. Lubricin is highly surface active molecule (e.g., holds onto water), that acts primarily as a potent cytoprotective, anti-adhesive and boundary lubricant. It is characterized by a long, central mucin-like domain located between terminal protein domains that allow the molecule to adhere and protect tissue surfaces. Its natural form, in all mammals investigated, contains multiple repeats of an amino acid sequence which is at least 50% identical to KEPAPTT (SEQ ID NO:3). Natural lubricin typically comprises multiple redundant forms of this repeat, but typically includes proline and threonine residues, with at least one threonine being glycosylated in most repeats. The threonine anchored O-linked sugar side chains are critical for lubricin's boundary lubricating function. The side chain moiety typically is a β(1-3)Gal-GalNAc moiety, with the β(1-3)Gal-GalNAc typically capped with sialic acid or N-acetylneuraminic acid. The polypeptide also contains N-linked oligosaccharides. The gene encoding naturally-occurring full length lubricin contains 12 exons, and the naturally-occurring MSF gene product contains 1,404 amino acids (including the secretion sequence) with multiple polypeptide sequence homologies to vitronectin including hemopexin-like and somatomedin-like regions. Centrally-located exon 6 contains 940 residues. Exon 6 encodes the repeat rich, 0-glycosylated mucin domain.

The amino acid sequence of the protein backbone of lubricin may differ depending on alternative splicing of exons of the human MSF gene. This robustness against heterogeneity was exemplified when researchers created a recombinant form of lubricin missing 474 amino acids from the central mucin domain, yet still achieved reasonable, although muted, lubrication (Flannery et al., Arthritis Rheum 2009; 60(3):840-7). PRG4 has been shown to exist not only as a monomer but also as a dimer and multimer disulfide-bonded through the conserved cysteine-rich domains at both N- and C-termini. Lubris, LLC has developed a full-length recombinant form of human lubricin. The molecule is expressed using the Selexis Chinese hamster ovary cell line (CHO-M), with a final apparent molecular weight of 450-600 kDa, with polydisperse multimers frequently measuring at 2,000 kDa or more, all as estimated by comparison to molecular weight standards on SDS tris-acetate 3-8% polyacrylamide gels. Of the total glycosylations, about half comprise two sugar units (GalNAc-Gal), and half three sugar units (GalNAc-Gal-Sialic acid). This method of recombinant human PRG4 production is provided in International Patent Application No. PCT/US014/061827.

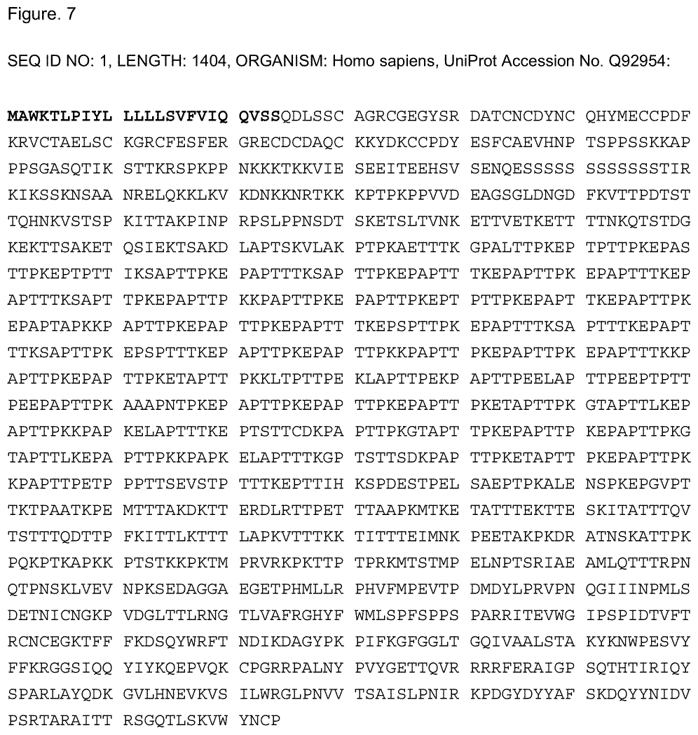

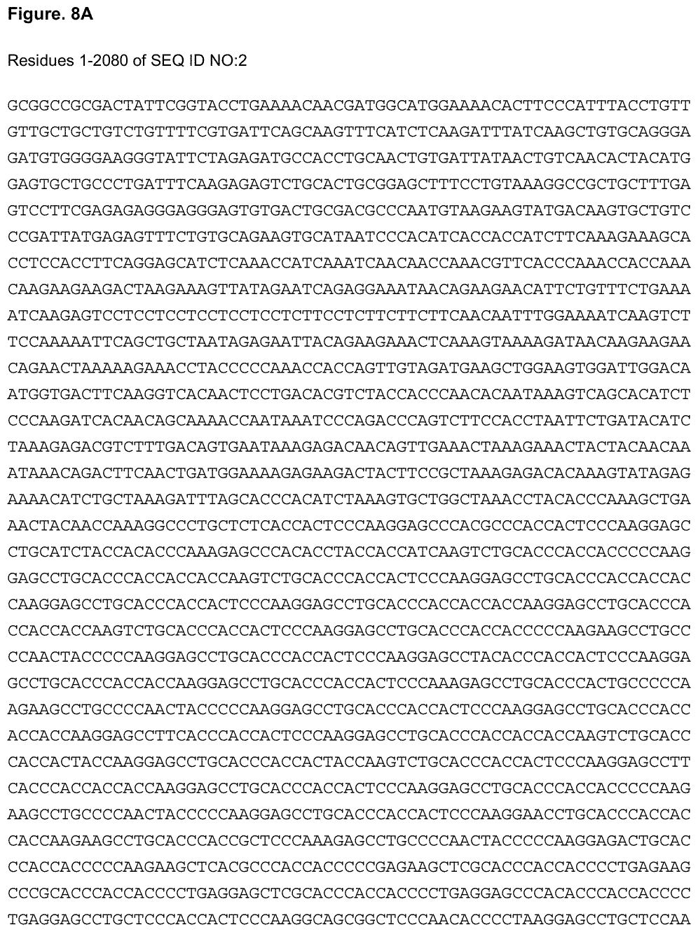

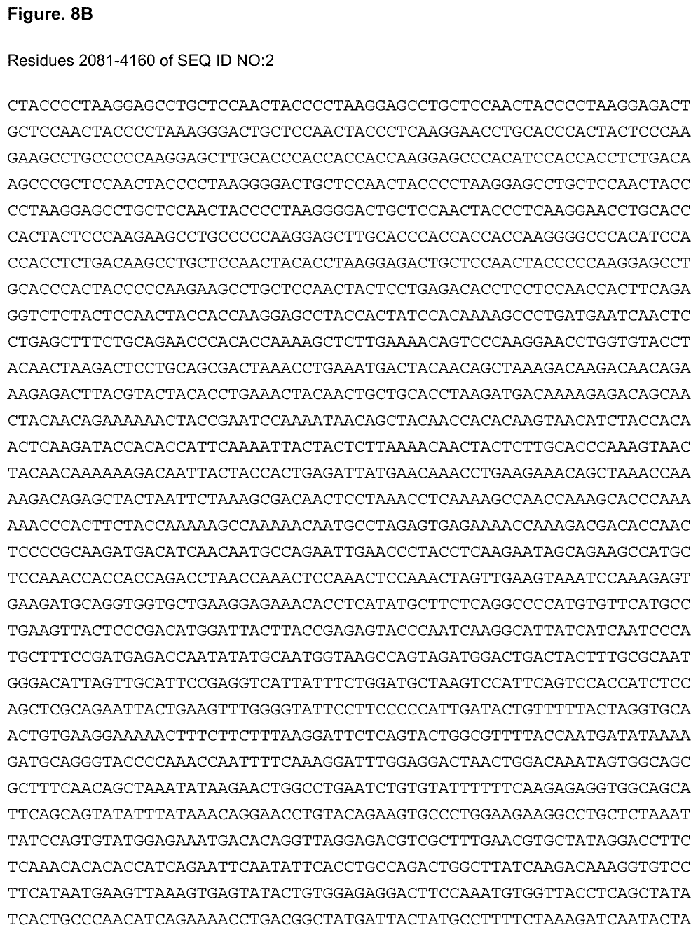

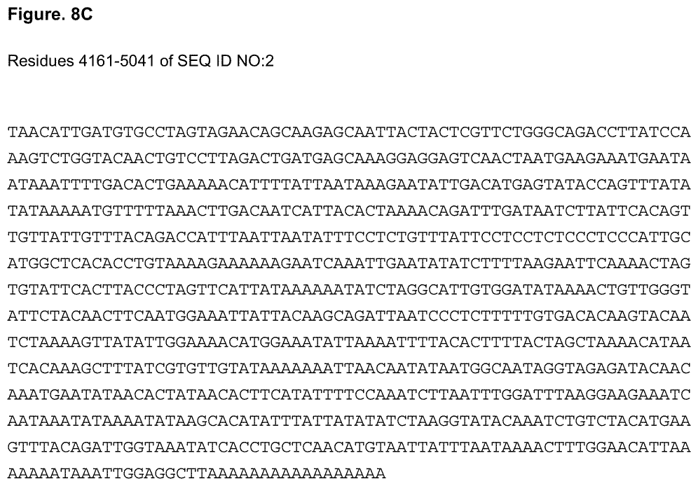

Any one or more of various native and recombinant PRG4 proteins and isoforms may be utilized in the various embodiments described herein. For instance, U.S. Pat. Nos. 6,433,142; 6,743,774; 6,960,562; 7,030,223, and 7,361,738 disclose how to make various forms of human PRG4 expression product, each of which is incorporated herein by reference. Preferred for use in the practice of the invention is full length, glycosylated, recombinant PRG4, or lubricin, expressed from CHO cells. This protein comprises 1,404 amino acids (see FIG. 7; SEQ ID NO:1) including a central exon comprising repeats of the sequence KEPAPTT (SEQ ID NO: 3) variously glycosylated with O-linked β (1-3) Gal-GalNAc oligosaccharides, and including N and C-terminal sequences with homology to vitronectin. The molecule is polydisperse with the glycosylation pattern of individual molecules varying, and can comprise monomeric, dimeric, and multimeric species.

As used herein, the term “PRG4” is used interchangeably with the term “lubricin.” Broadly, these terms refer to any functional isolated or purified native or recombinant properly glycosylated PRG4 proteins, homologs, functional fragments, isoforms, and/or mutants thereof. All useful molecules comprise the sequence encoded by exon 6, or homologs or truncated versions thereof, for example, versions with fewer repeats within this central mucin-like KEPAPTT-repeat domain (SEQ ID N0:3), together with O-linked glycosylation. All useful molecules also comprise at least the biological active portions of the sequences encoded by exons 1-5 and 7-12, i.e., sequences responsible for imparting to the molecule its affinity for ECM and endothelial surfaces. In certain embodiments, a preferred PRG4 protein has an average molar mass of between 50 kDa and 500 kDa, preferably between 224 to 467 kDa, comprising one or more biologically active portions of the PRG4 protein, or functional fragments, such as a lubricating fragment, or a homolog thereof. In a more preferred embodiment, a PRG4 protein comprises monomers of average molar mass of between 220 kDa to about 280 kDa.

In some embodiments, functional or biologically active PRG4 fragments and homologs are contemplated that have at least 50%, 55%, 60%, 65%, 70%, 75%, 80%, 85%, 90%, 95%, or 98% amino acid sequence identity with SEQ ID NO:1 or with the sequences encoded by exons 1-5 and 7-12 of PRG4. In some embodiments, functional or biologically active PRG4 fragments and homologs are contemplated that have at least 50%, 55%, 60%, 65%, 70%, 75%, 80%, 85%, 90%, 95%, or 98% sequence identity with residues 25-1404 of SEQ ID NO:1. In another embodiment, the PRG4 is recombinant human lubricin. In another embodiment, the PRG4 has the amino acid sequence of SEQ ID NO:1. In another embodiment, the PRG4 has the amino acid sequence of residues 25-1404 SEQ ID NO:1.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino acid or nucleic acid sequence). The percent identity between the two sequences is a function of the number of identical positions shared by the sequences (i.e., % homology=(# of identical positions/total # of positions)times 100). The determination of percent homology between two sequences can be accomplished using a mathematical algorithm. A non-limiting example of a mathematical algorithm utilized for the comparison of two sequences is the algorithm of Karlin and Altschul, (1990) Proc. Natl. Acad. Sci. USA, 87:2264-68, modified as in Karlin and Altschul, (1993) Proc. Natl. Acad. Sci. USA, 90:5873-77. Such an algorithm is incorporated into the NBLAST and XBLAST programs of Altschul et al., (1990) J. Mol. Biol., 215:403-10. BLAST nucleotide searches can be performed with the NBLAST program, score=100, wordlength=12. BLAST protein searches can be performed with the XBLAST program, score=50, wordlength=3. To obtain gapped alignments for comparison purposes, Gapped BLAST can be utilized as described in Altschul et al., (1997) Nucleic Acids Research, 25(17):3389-3402. When utilizing BLAST and Gapped BLAST programs, the default parameters of the respective programs (e.g., XBLAST and NBLAST) can be used.

In some embodiments, functional PRG4 fragments and homologs are contemplated that have at least 50%, 55%, 60%, 65%, 70%, 75%, 80%, 85%, 90%, 95%, or 98% activity as compared with native PRG4, e.g., biological activity.

Methods for isolation, purification, and recombinant expression of a PRG4 protein are well known in the art. In certain embodiments, the method starts with cloning and isolating mRNA and cDNA encoding PRG4 proteins or isoforms using standard molecular biology techniques, such as PCR or RT-PCR. The isolated cDNA encoding the PRG4 protein or isoform is then cloned into an expression vector, and expressed in a host cell for producing recombinant PRG4 protein, and isolated from the cell culture supernatant. A method for production of recombinant human PRG4 is provided in International Patent Application No. PCT/US014/061827.

The function of PRG4 heretofore has been almost entirely associated with prevention of wear between articulating joints and lubrication of interfacing tissues such as between the surface of the eye and eyelid. The functional importance of PRG4 in joint maintenance has been shown by mutations that cause the camptodactyly-arthropathy-coxa vara-pericarditis (CACP) disease syndrome in humans. CACP is manifest by camptodactyly, noninflammatory arthropathy, and hypertrophic synovitis, with coxa vara deformity, pericarditis, and pleural effusion. Also, in PRG4-null mice, cartilage deterioration and subsequent joint failure were observed. Therefore, PRG4 expression is a necessary component of healthy synovial joints. However, use of a systemic boundary lubricant such as PRG4 protein to treat gout as described in the present invention to Applicants' knowledge has not been previously suggested.

### Patient Population to be Treated

Patients suffering from gout can be treated by administration of PRG4 or a functional or biologically active fragment thereof. Patients suffering from pain associated with gout can also be treated by administration of PRG4 or a functional fragment thereof. In one embodiment, the patient is a mammal. In a particular embodiment, the patient is a human. The patient may also be another mammal, such as a cat, dog, horse, cow, pig or sheep.

In one embodiment, when a human is treated, the PRG4 is recombinant human PRG4. In another embodiment, when a dog is treated, the PRG4 is dog or human recombinant PRG4. In another embodiment, when a cat is treated, the PRG4 is cat or human recombinant PRG4. In another embodiment, when a horse is treated, the PRG4 is horse or human recombinant PRG4. In another embodiment, when a cow is treated, the PRG4 is cow or human recombinant PRG4. In another embodiment, when a pig is treated, the PRG4 is pig or human recombinant PRG4. In another embodiment, when a sheep is treated, the PRG4 is sheep or human recombinant PRG4.

### Administration of PRG4

While PRG4 is produced naturally within the body, the effects of the invention are observed when exogenous PRG4 is administered to the patient. Accordingly, in one embodiment, the PRG4 administered to the patient is exogenous human PRG4, while in another embodiment, the PRG4 administered to the patient is recombinant human PRG4 (rhPRG4). In another embodiment, rhPRG4 has the sequence of SEQ ID NO:1 or residues 25-1404 of SEQ ID NO:1.

The amount of PRG4 administered will depend on variables such as the severity of symptoms including the level of joint pain the patient experiences, the seriousness of gout (i.e., level of crystal deposition or inflammation in the joint(s)), the overall health of the patient, the pharmaceutical formulation, and the route of administration. The initial dosage can be increased beyond the upper level in order to rapidly achieve the desired blood-level or tissue level. Alternatively, the initial dosage can be smaller than the optimum, and the dosage may be progressively increased during the course of treatment. Alternatively, the initial dosage can be smaller than the optimum, and the dosage may be progressively increased during the course of treatment. The optimal dose can be determined by routine experimentation.

In one embodiment, the PRG4 is administered in an amount that is insufficient to provide boundary lubrication, but sufficient to treat joint pain or allodynia. In one embodiment, the PRG4 is administered in an amount that is insufficient to provide boundary lubrication, but sufficient to reduce inflammation associated with gout. Accordingly, in some embodiments, a therapeutically effective amount of PRG4 for administration according to the invention is in the range of 0.1 μg/kg to 4000 μg/kg, or 0.1 μg/kg to 1000 μg/kg, or 0.1 μg/kg to 100 μg/kg, or 0.1 to 50 μg/kg. In some embodiments, the therapeutically effective amount of PRG4 administered is in the range of 0.1 mg/kg to 100 mg/kg, or 1 mg/kg to 100 mg/kg, or 1 mg/kg to 10 mg/kg. The PRG4 administered may also be in a range of 0.1 μg/mL to 30 mg/mL, or 1 μg/mL to 10 mg/mL, or 10 μg/mL to 1 mg/mL. In some embodiments, PRG4 is administered at concentrations no greater than 60 μg/mL. In some embodiments, PRG4 is administered in small volumes of 1 to 100 μL per dose.

In further embodiments, lubricin is administered locally or systemically in an amount sufficient to achieve a concentration of lubricin in a synovial fluid of a joint of a subject of at least 50 μg/ml, at least 100 μg/ml, at least 150 μg/ml, at least 200 μg/ml, at least 250 μg/ml, at least 300 μg/ml, at least 350 μg/ml, at least 400 μg/ml, at least 450 μg/ml, at least 500 μg/ml, at least 550 μg/ml, at least 600 μg/ml, at least 650 μg/ml, at least 750 μg/ml, at least 800 μg/ml, at least 850 μg/ml, at least 900 μg/ml, at least 950 μg/ml, or at least 1000 μg/ml. It is contemplated that to achieve a concentration of lubricin in the synovial fluid of a joint of a subject, lubricin must be administered to the subject at a concentration higher than the desired concentration of lubricin in the synovial fluid. In certain embodiments, a total amount of 2 mg to 10 mg of lubricin is administered per dose, e.g., 2 mg to 10 mg, 2 mg to 5 mg, 2 mg to 3 mg, 3 mg to 4 mg, 4 mg to 5 mg, 5 mg to 6 mg, 6 mg to 7 mg, 7 mg to 8 mg, 8 mg to 9 mg, 9 mg to 10 mg, or 5 mg to 10 mg. In certain embodiments, more than 10 mg of lubricin is administered per dose. In some embodiments, the lubricin is administered intra-articularly to the joint to achieve the desired concentration of PRG4 in the synovial fluid. The PRG4 may also be administered intravenously to achieve the desired concentration of PRG4 in the synovial fluid. It is contemplated in this invention that the dose of PRG4 used for intravenous administration is at least 1.5 fold, or at least 2 fold, or at least 3 fold, or at least 4 fold, or at least 5 fold, or at least 10 fold higher than dose used for intra-articular administration. For example, in one embodiment, PRG4 is administered to a patient suffering from gout wherein PRG4 is administered in the amount 0.05-1.50 mg/kg.

The current invention contemplates that PRG4 may be administered to the patient suffering from gout systemically or locally. Local administration, for example, intra-articular administration into an affected joint or injection into an affected area is contemplated by the invention. In some embodiments, injection directly into an affected joint such as a hip, shoulder, elbow, knee, toe, finger, ankle, or wrist is contemplated. In some other embodiments, injection into an affected area such as the instep or heel of the foot is contemplated. Accordingly, a therapeutically effective amount of PRG4 for local administration according to the invention may be in the range of 0.1 μg/kg to 4000 μg/kg, or 0.1 μg/kg to 1000 μg/kg, or 0.1 μg/kg to 100 μg/kg, or 0.1 to 50 μg/kg. PRG4 administered may also be in an range of 0.1 μg/mL to 30 mg/mL, or 1 μg/mL to 10 mg/mL, or 10 μg/mL to 1 mg/mL. PRG4 administered may also be in an amount of 2 mg to 10 mg, 2 mg to 5 mg, 5 mg to 10 mg or greater than 10 mg. These administrations may be carried out every day, every other day, every three days, every four days, every five days, every six days, once weekly, once every other week, once every third week, or once monthly per treatment cycle.

Systemic administration of PRG4 is also contemplated by some embodiments of the invention. For example, PRG4 may be systemically administered in an enteral manner, such as oral, rectal, sublingual, sublabial, or buccal delivery. PRG4 may be systemically administered in a parenteral manner, such as nasal, by inhalation, intravenous, intramuscular, subcutaneous, intradermal, or transmucosal delivery.

A preferred route of systemic administration of PRG4 contemplated herein is intravenous administration. The optimal dose can be determined by routine experimentation depending on variables such as the level of joint pain the patient is experiencing, the seriousness of gout, the overall health of the patient, and the pharmaceutical formulation. For systemic administration, a dose between 0.1 mg/kg and 100 mg/kg, alternatively between 0.5 mg/kg and 50 mg/kg, alternatively, between 1 mg/kg and 25 mg/kg, alternatively between 2 mg/kg and 10 mg/kg, alternatively between 5 mg/kg and 10 mg/kg, alternatively between 0.05-1.50 mg/kg is administered and may be given, for example, once daily, once weekly, twice weekly, three times weekly, once every other week, once every third week, or once monthly per treatment cycle.

For therapeutic use, the PRG4 administered is preferably combined with a pharmaceutically acceptable carrier. As used herein, “pharmaceutically acceptable carrier” means buffers, carriers, and excipients suitable for use in contact with the tissues of human beings and animals without excessive toxicity, irritation, allergic response, or other problem or complication, commensurate with a reasonable benefit/risk ratio. The carrier(s) should be “acceptable” in the sense of being compatible with the other ingredients of the formulations and not deleterious to the recipient. Pharmaceutically acceptable carriers include buffers, solvents, dispersion media, coatings, isotonic and absorption delaying agents, and the like, that are compatible with pharmaceutical administration. Suitable carriers include phosphate buffered saline at concentrations ranging from 1 μg/ml to 1000 μg/ml, and more preferably 100-500 μg/ml. Suitable carriers may also include physiological saline, bacteriostatic water, Cremophor ELTM (BASF, Parsippany, N.J.) or phosphate buffered saline (PBS), optionally in admixture with surfactants such as polysorbates. Suitable carriers may also include a solvent or dispersion medium containing, for example, water, ethanol, polyol (for example, glycerol, propylene glycol, and liquid polyethylene glycol), and suitable mixtures thereof. The carrier should be stable under the conditions of manufacture and storage, and should be preserved against microorganisms. The use of carriers for pharmaceutically active substances is known in the art. For example, see Remington's Pharmaceutical Sciences, 18th ed. (Mack Publishing Company, 1990).

Useful formulations can be prepared by methods well known in the pharmaceutical arts. For example, see Remington's Pharmaceutical Sciences, 18th ed. (Mack Publishing Company, 1990). Formulation components suitable for parental administration include a sterile diluent such as water for injection, saline solution, fixed oils, polyethylene glycol, glycerin, propylene glycol or other synthetic solvents; antibacterial agents such as benzyl alcohol or methyl paraben; antioxidants such as ascorbic acid or sodium bisulfite; chelating agents such as EDTA; buffers such as acetates, citrates or phosphates; and agents for the adjustment of tonicity such as sodium chloride or dextrose. Lubricin for administration can be present in a dosage unit form and can be prepared by any suitable method and should be formulated to be compatible with its intended route of administration.

PRG4 for administration should be formulated to be compatible with its intended route of administration, for example, intra-articular (IA), intravenous (IV), intramuscular, subcutaneous, intradermal, intranasal, transdermal, topical, transmucosal, oral and rectal administration. The formulation of PRG4 can be presented in a dosage unit form and prepared by any suitable method known in the art. For example, formulation components suitable for parenteral administration include a sterile diluent such as water for injection, saline solution, fixed oils, polyethylene glycols, glycerine, propylene glycol or other synthetic solvents; antibacterial agents such as benzyl alcohol or methyl paraben; antioxidants such as ascorbic acid or sodium bisulfite; chelating agents such as EDTA; buffers such as acetates, citrates or phosphates; and agents for the adjustment of tonicity such as sodium chloride or dextrose.

Pharmaceutical formulations for PRG4 preferably are sterile. Sterilization can be accomplished, for example, by filtration through sterile filtration membranes. Where the composition is lyophilized, filter sterilization can be conducted prior to or following lyophilization and reconstitution. Aqueous solutions may be packaged for use as-is, or lyophilized, the lyophilized preparation being combined with a sterile aqueous carrier prior to administration. The pH of the preparations typically is between 3 and 11, more preferably between 5 and 9 or between 6 and 8, and most preferably between 7 and 8, such as 7 to 7.5. Formulated PRG4 for administration may be packaged in multiple single dose units, each containing a fixed amount of the above-mentioned agent or agents, such as in a sealed package of tablets or capsules.

Pharmaceutical compositions containing PRG4, such as those disclosed herein, can be presented in a dosage unit form and can be prepared by any suitable method. A pharmaceutical composition should be formulated to be compatible with its intended route of administration. Examples of routes of administration are intravenous (IV), intradermal, inhalation, transdermal, topical, transmucosal, and rectal administration. The pharmaceutical compositions are intended for parenteral, intranasal, topical, oral, or local administration, such as by a transdermal means, for therapeutic treatment. The pharmaceutical compositions can be administered parenterally (e.g., by intravenous, intramuscular, or subcutaneous injection), or by oral ingestion, or by topical application or intraarticular injection at areas affected by gout such as the knee, ankle, finger joint, or elbow. Additional routes of administration include intravascular, intra-arterial, intratumor, intraperitoneal, intraventricular, intraepidural, as well as nasal, ophthalmic, intrascleral, intraorbital, rectal, topical, or aerosol inhalation administration.

The invention provides compositions for parenteral administration that comprise the above mentioned agents dissolved or suspended in an acceptable carrier, preferably an aqueous carrier, e.g., water, buffered water, saline, PBS, and the like. The compositions may contain pharmaceutically acceptable auxiliary substances as required to approximate physiological conditions, such as pH adjusting and buffering agents, tonicity adjusting agents, wetting agents, detergents and the like. The invention also provides compositions for oral delivery, which may contain inert ingredients such as binders or fillers for the formulation of a tablet, a capsule, and the like. Furthermore, this invention provides compositions for local administration, which may contain inert ingredients such as solvents or emulsifiers for the formulation of a cream, an ointment, and the like.

A preferred route of administration for PRG4 IV infusion. Useful formulations can be prepared by methods well known in the pharmaceutical art. For example, see Remington's Pharmaceutical Sciences, 18th ed. (Mack Publishing Company, 1990). Formulation components suitable for parenteral administration include a sterile diluent such as water for injection, saline solution, fixed oils, polyethylene glycols, glycerine, propylene glycol or other synthetic solvents; antibacterial agents such as benzyl alcohol or methyl paraben; antioxidants such as ascorbic acid or sodium bisulfite; chelating agents such as EDTA; buffers such as acetates, citrates or phosphates; and agents for the adjustment of tonicity such as sodium chloride or dextrose.

### EXAMPLES

### Example 1. The Impact of Anti-TLR2 Antibody and rhPRG4 Treatment on MSU Phagocytosis by THP-1 Macrophages

Differentiation of THP-1 monocytes (ATCC, USA) into macrophages was performed as previously described (Park E K et al., Inflamm Res 2007; 56:45-50.). A THP-1 monocyte cell line was obtained from American Type Culture Collection (ATCC, USA). Cells were cultured to a density of 1.5×10⁶ cells/mL in 75 cm flask in RPMI 1640 medium supplemented with 10% heat-inactivated fetal bovine serum (FBS), 10 mM HEPES, 2 mM glutamine, 100 U/L Penicillin and 100 μg/ml streptomycin and maintained at 37° C. under 5% CO₂. In sterile 12 well plates (Corning, Sigma Aldrich, USA), 500,000 cells in 2 ml RPMI 1640 media were differentiated into macrophages by incubation with phorbol 12-myristate-13-acetate (PMA; Sigma Aldrich) to a final concentration of 5 ng/ml for 48 hours. Subsequently, media supernatants were removed and wells were washed three times with sterile PBS to remove any unattached cells and new RPMI 1640 media was added.

Following differentiation of THP-1 monocytes into macrophages, cells were treated with endotoxin-free MSU crystals (100 μg/mL; Invivogen, USA) in the absence or presence of anti-TLR2 antibody or rhPRG4 for 6 hours at 37° C. Subsequently, media supernatants were removed and adhered macrophages were harvested via trypsinization, pelleted and washed three times with PBS. The phagocytosis of MSU crystals was determined by analyzing the side scatter (SSc) changes using a flow cytometer (Guava easyCyte Flow Cytometer, EMD Millipore, USA). The SSc changes were determined across three independent experiments using the same acquisition parameters. Anti-TLR2 antibody (MAB 2616, R&D Systems, USA) was pre-incubated with macrophages for 2 hours at a final concentration of 20 μg/ml. rhPRG4 treatment was performed to a final concentration of 200 μg/mL. rhPRG4 is an endotoxin-free full-length product produced by CHO-M cells (Lubris, Framingham, Mass., USA) (Samson M L et al., Exp Eye Res 2014; 127C:14-19). MSU phagocytosis by THP-1 macrophages was quantitatively analyzed by estimating the percentage of cells whose SSc values were higher than a pre-defined threshold value.

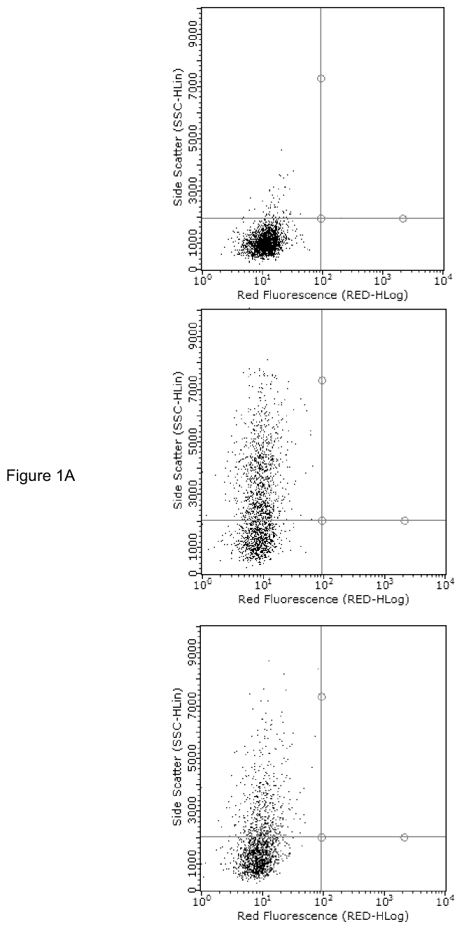

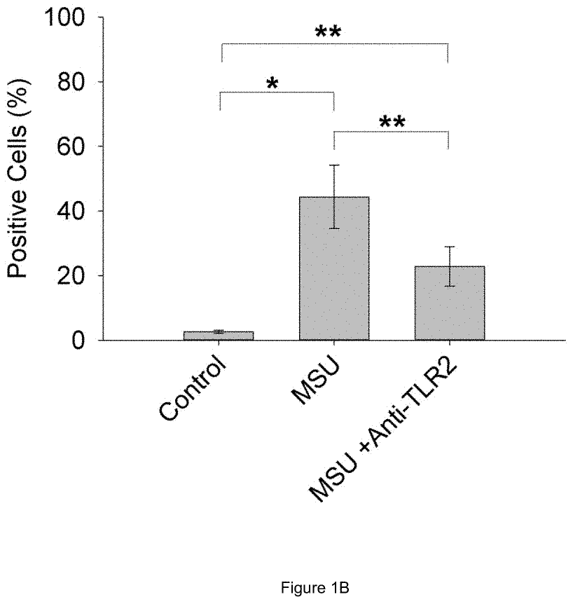

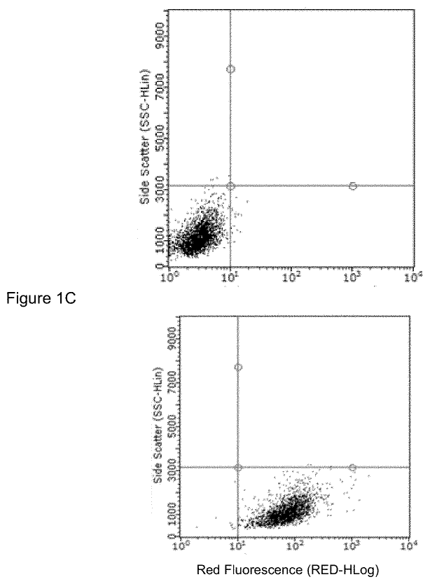

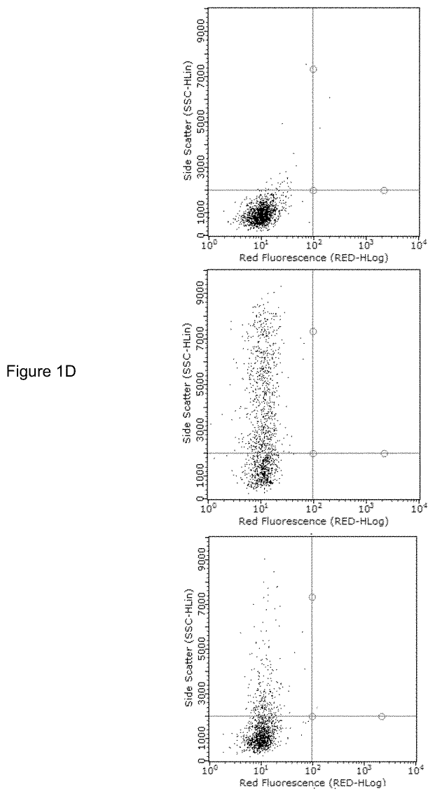

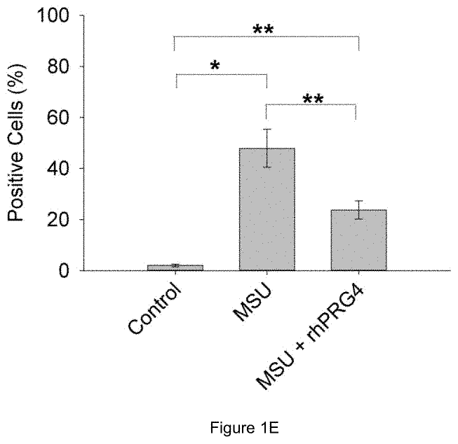

Representative flow cytometry scatter plots of control untreated macrophages, MSU-treated and MSU+anti-TLR2 antibody-treated macrophages are shown in FIG. 1A. Control macrophages were localized in the lower left quadrant of the scatter plot (FIG. 1A, top image). MSU phagocytosis resulted in an increase in SSc of the macrophage cell population (FIG. 1A, middle image). Pre-treatment with an anti-TLR2 antibody reduced the upward shift in SSc of the cell population (FIG. 1A, bottom image). Quantitative analysis of MSU phagocytosis by THP-1 macrophages in the absence or presence of anti-TLR2 antibody is shown in FIG. 1B. The percentage of positive macrophages in the MSU-treated group was significantly higher than the percentage of positive macrophages in the control untreated group (p<0.001). The percentage of positive macrophages in the MSU+anti-TLR2 antibody-treated group was significantly lower than percentage of positive macrophages in MSU-treated group (p<0.01) and higher than in the control untreated group (p<0.01).

In another experiment, rhPRG4 was labeled with Rhodamine using a commercially available labeling kit according to manufacturer's recommendation (Pierce NHS—Rhodamine Antibody Labeling Kit, ThermoFisher Scientific, USA). Following differentiation of THP-1 monocytes into macrophages, cells were treated with Rhodamine-rhPRG4 (20 μg/mL) for 6 hours at 37° C. Subsequently, macrophages were collected as described above and cell associated fluorescence was analyzed using flow cytometry. A threshold was set at red fluorescence intensity=10, and cells that displayed fluorescence intensity higher than the threshold were counted as positively associating with Rhodamine-rhPRG4.

Rhodamine-labeled rhPRG4 associated with THP-1 macrophages as evidenced by a shift in the macrophage cell population from the lower left quadrant to the lower right quadrant in the flow cytometry plot (FIG. 1C). Representative flow cytometry plots of control untreated THP-1 macrophages, MSU-treated and MSU+rhPRG4 treated macrophages are shown in FIG. 1D. rhPRG4 treatment reduced the SSc shift of the macrophage cell population, attributed to MSU phagocytosis (FIG. 1C, bottom image). Quantitative analysis of MSU phagocytosis by THP-1 macrophages in the absence or presence of rhPRG4 is shown in FIG. 1E. The percentage of positive macrophages in the MSU-treated groups was higher than in the control untreated group (p<0.004 The percentage of positive macrophages in the MSU+rhPRG4 treated group was significantly lower than in MSU-treated group (p<0.01) and higher than the control group (p<0.01).

### Example 2. Impact of rhPRG4 Treatment on MSU-Induced Pro-Inflammatory Cytokines and Chemokines Gene Expression in THP-1 Macrophages

Following differentiation of THP-1 monocytes, macrophages were treated with MSU (100 μg/mL) in the absence or presence of rhPRG4 (25, 50, 100 and 200 μg/mL) for 24 hours. Following treatment, total RNA was extracted using trizol reagent (Thermo Fisher Scientific), and RNA concentrations were determined with a NanoDrop ND-2000 spectrophotometer (NanoDrop Technologies, USA). cDNA was synthesized using Transcriptor First Strand cDNA Synthesis Kit (Roche, USA). Quantitative PCR (qPCR) was performed on Applied Biosystems Step One Plus Real-Time PCR System (Thermo Fisher Scientific, USA) using TaqMan Fast Advanced Master Mix (Life Technologies, USA). The genes of interest included IL-1β (Hs00174097_m1, ThermoFisher Scientific), TNF-α (Hs01113624_g1, ThermoFisher Scientific), MCP-1 (Hs00234140_m1, ThermoFisher Scientific) and IL-8 (Hs00174103_m1, ThermoFisher Scientific). The cycle threshold (Ct) value of target genes was normalized to the Ct value of GAPDH (Hs02758991_g1; Thermo Fisher Scientific) in the same sample, and the relative expression was calculated using the 2^{-ΔΔct} method (Livak K J et al., Methods 2001; 25:402-8). Data is presented as fold target gene expression compared to untreated control. Data represents the average of 3 independent experiments with duplicate wells per treatment.

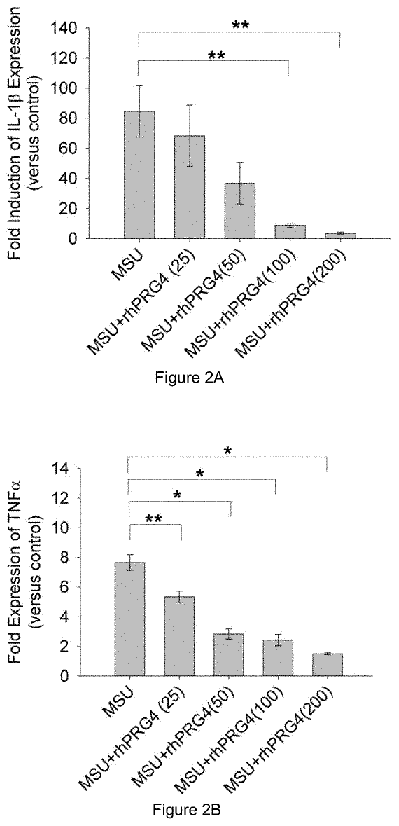

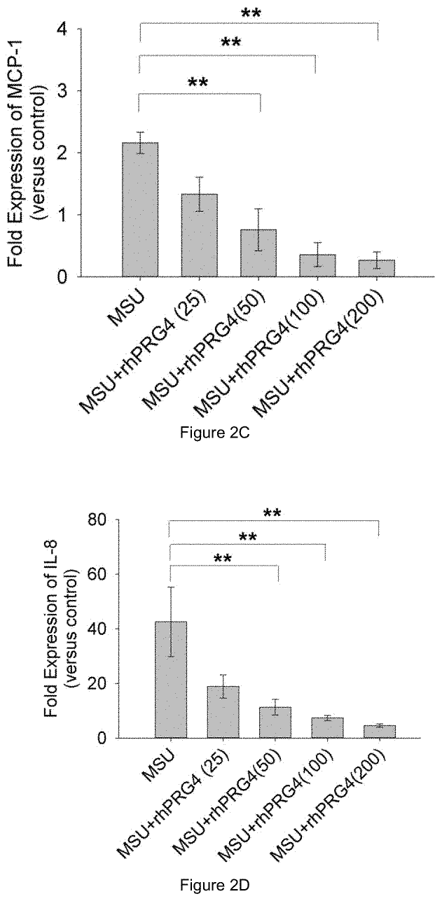

Results show that rhPRG4 treatment inhibits MSU-induced IL-1β, TNF-α, MCP-1 and IL-8 gene expression in THP-1 macrophages. Induction of IL-1β, TNF-α, MCP-1 and IL-8 gene expression in differentiated THP-1 macrophages by MSU crystals and the impact of rhPRG4 treatment is shown in FIGS. 2A-D. MSU crystals significantly induced IL-1β gene expression compared to untreated macrophages (FIG. 2A). rhPRG4 (100 and 200 μg/ml) treatments reduced IL-1β gene expression in THP1 macrophages following incubation with MSU crystals (p<0.01). Likewise, MSU crystals significantly induced TNF-α gene expression compared to untreated macrophages (FIG. 2B). rhPRG4 (50, 100 and 200 μg/ml) treatments reduced TNF-α gene expression compared to the MSU alone group (p<0.01; p<0.001; p<0.001). MSU crystals significantly induced chemokine MCP-1 and IL-8 gene expression compared to untreated macrophages (p<0.01) (FIGS. 2C and 2D). rhPRG4 (50, 100 and 200 μg/ml) treatments reduced MCP-1 and IL-8 gene expression compared to MSU alone group (p<0.01).

### Example 3. Impact of rhPRG4 Treatment on MSU-Induced Pro-Inflammatory Cytokines and Chemokines Production by THP-1 Macrophages

Following differentiation of THP-1 monocytes, macrophages were treated with MSU (100 μg/mL) in the absence or presence of rhPRG4 (100 and 200 μg/mL) for 24 hours. Subsequently, media supernatants were collected and media concentrations of IL-β, TNF-α, MCP-1 and IL-8 were determined using commercially-available ELISA kits (R&D Systems, USA). Data is presented as Percent of cytokine and chemokine concentration in the untreated control macrophages. Data represents the mean±S.D. of 3 independent experiments with duplicate wells per group.

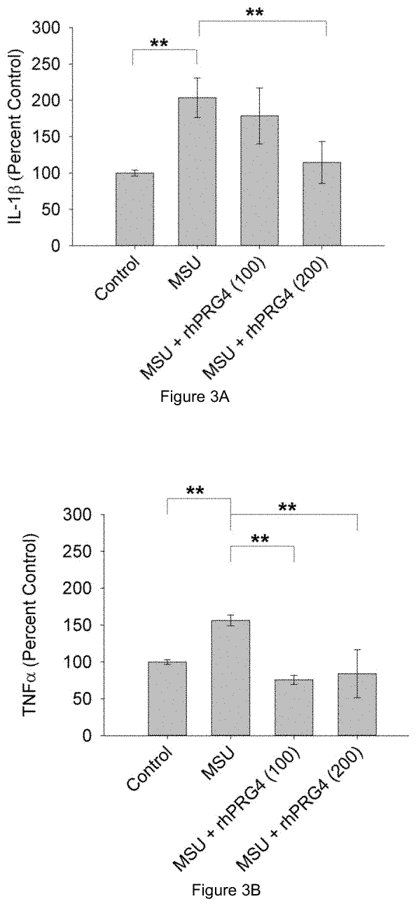

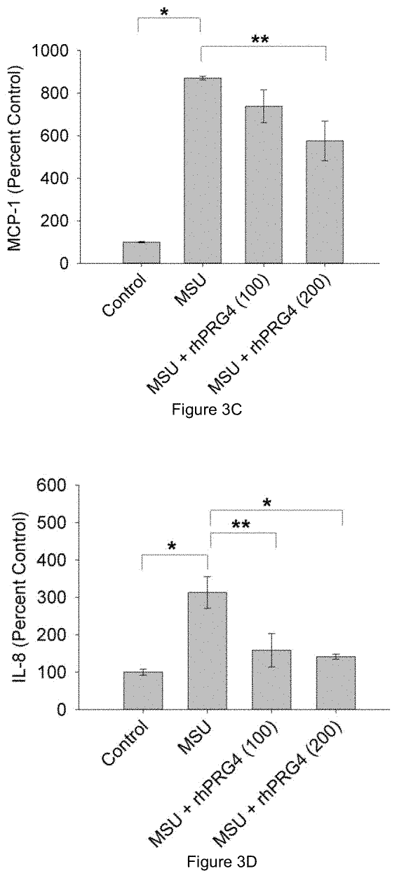

Results show that rhPRG4 treatment inhibits MSU-induced IL-β, TNF-α, MCP-1 and IL-8 production by THP-1 macrophages. Pro-inflammatory cytokines and chemokines concentrations in media supernatants from untreated macrophages and MSU-treated macrophages in the absence or presence of rhPRG4 is shown in FIG. 3. Treatment with MSU crystals increased IL-1β media concentrations compared to controls (p<0.01) (FIG. 3A). rhPRG4 (200 μg/ml) treatment significantly reduced MSU-induced IL-1β production by macrophages (p<0.01). MSU crystals significantly increased TNF-α production by THP-1 macrophages (p<0.01) (FIG. 3B). rhPRG4 (100 and 200 μg/ml) treatments significantly reduced MSU-induced TNF-α production by macrophages (p<0.01). MSU crystals significantly induced chemokines MCP-1 and IL-8 production by macrophages (p<0.001) (FIGS. 3C and 3D). rhPRG4 (200 μg/ml) treatment significantly reduced MSU-induced MCP-1 production by macrophages (p<0.01), while rhPRG4 (100 and 200 μg/ml) treatments significantly reduced IL-8 production by macrophages (p<0.01; p<0.001).

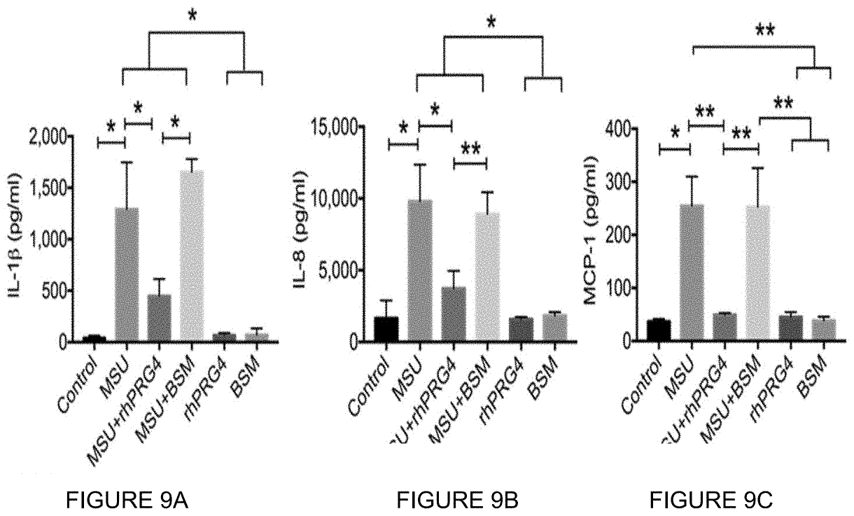

In yet another experiment, differentiated THP-1 macrophages were treated with MSU in the amount 0.1 mg/ml. Bovine submaxillary mucin (BSM) (>1000 kDa, 25 μg/mL) was used as a prototypical mucin control which did not alter MSU-induced IL-1β, IL-8 or MCP-1 production. Macrophages co-treated with rhPRG4 (240 kDa; 100 ug/mL) showed significantly decreased amounts of IL-1β, IL-8 and MCP-1 in conditioned media. (See FIGS. 9A-C). rhPRG4 and BSM had no effect on basal cytokine production by THP-1 macrophages.

### Example 4. Impact of rhPRG4 Treatment on MSU-Induced IL-1β Production by Prg4^{+/+} and Prg4^{-/-} Peritoneal Macrophages

Isolation of murine peritoneal macrophages was performed as previously described (Livak K J et al., Methods 2001; 25:402-8). A total of 5 Prg4^{+/+} and 5 Prg4^{-/-} mice were euthanized. Subsequently, the abdomen of each mouse was soaked with 70% alcohol and a small incision was made along the midline with scissors. Using blunt dissection, the abdominal skin was retracted to expose the intact peritoneal wall. A 27 G needle attached to a 10 ml syringe filled with sterile cold PBS was inserted through the peritoneal wall at the midline and injected into each mouse, aspirated slowly from the peritoneum, and peritoneal macrophages cells were collected. Subsequently, cells were centrifuged at 10,000 rpm and 4° C. for 10 min. Pelleted cells were re-suspended in RPMI 1640 medium supplemented with 10% FBS and 1% Penicillin/Streptomycin.

Murine peritoneal macrophages were plated onto sterile chamber slides at a concentration of 1.3×10⁶ cells/well. Peritoneal macrophages were allowed to adhere by incubation at 37° C. for 24 hours. Following incubation, media and non-adherent cells were removed and fresh media was added. Treatments included untreated control cells, MSU (100 μg/mL) treatment, MSU (100 μg/mL)+rhPRG4 (200 μg/mL) treatment, and MSU (100 μg/mL)+anti-TLR2 antibody (20 μg/mL) treatment. Following a 6-hour incubation, slides were washed once with PBS, then fixed with 4% formalin for 15 min. Subsequently, slides were washed with PBS and cells were permeabilized with 0.1% triton X100 for 10 min. After washing with PBS for three times, slides were mounted with DAPI mounting medium (Vector Lab, USA) and viewed under a microscope (Nikon E800). The number of intracellular MSU crystals in 6-9 areas for a total of 900 cells were counted and the total number of MSU crystals were reported. Data represents the mean±S.D. of 5 independent experiments.

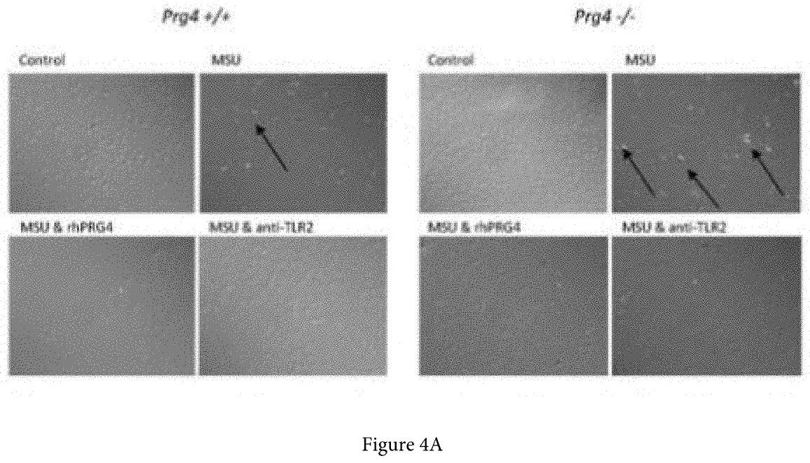

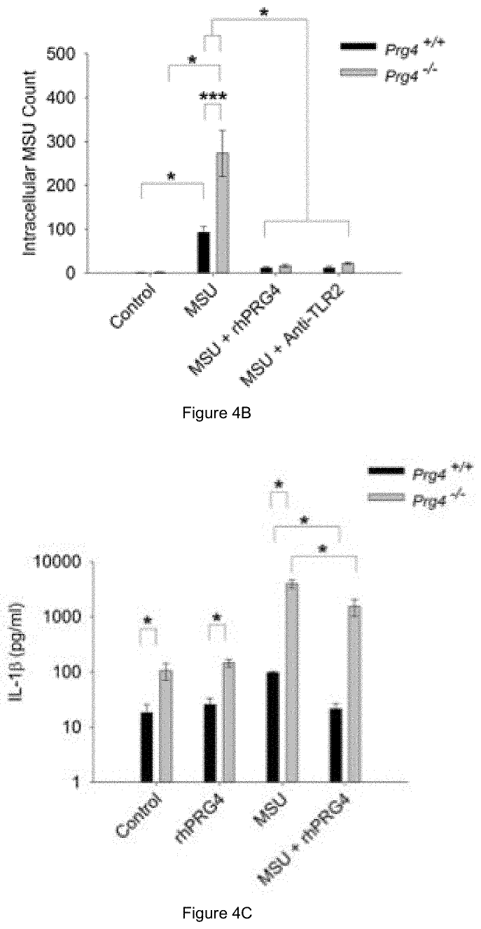

Representative images of DAPI-stained peritoneal macrophages from Prg4^{+/+} and Prg4^{-/-} mice following incubation with MSU crystals in the absence or presence of anti-TLR2 antibody or rhPRG4 are shown in FIG. 4A. MSU crystals appeared to have been internalized by Prg4^{+/+} and Prg4^{-/-} peritoneal macrophages, as indicated by arrows. An appreciable number of MSU crystals localized intracellularly in the anti-TL2 antibody or rhPRG4-treated macrophages from either genotype was not observed.

In another experiment, peritoneal macrophages were harvested from Prg4^{+/+} and Prg4^{-/-} mice as described above. Cells were centrifuged for 10 min at 4° C. and 10,000 rpm. The supernatant was discarded and the cell pellet was gently re-suspended in RPMI 1640 media supplemented with 10% FBS and 1% penicillin/streptomycin. Cells were counted with a hemacytometer and plated onto sterile cell culture plates at a concentration of 1.3×10⁶ cells/well. Peritoneal macrophages were allowed to adhere by culturing them for 24 hours at 37° C. Treatment groups included untreated control, rhPRG4 (200 μg/mL), MSU (100 μg/mL), and MSU (100 μg/mL)+rhPRG4 (200 μg/mL). Treatments were performed for 24 hours and media supernatants were assayed for IL-1β concentrations using a commercially-available ELISA kit (R&D Systems, USA). Data is presented as the mean of 5 independent experiments±standard error of the mean with at least triplicate wells per group.

Quantitation of MSU uptake by Prg4^{+/+} and Prg4^{-/-} peritoneal macrophages is shown in FIG. 4B. At 6 hours following incubation, MSU crystals were phagocytized by Prg4^{+/+} and Prg4^{-/-} peritoneal macrophages. MSU phagocytosis was significantly higher in Prg4^{-/-} peritoneal macrophages compared to Prg4^{+/+} peritoneal macrophages (p<0.05). Anti-TLR2 antibody and rhPRG4 treatments reduced MSU phagocytosis by Prg4^{+/+} and Prg4^{-/-} macrophages (p<0.001). There was no significant difference in MSU phagocytosis by macrophages of either genotype in the presence of anti-TLR2 antibody or rhPRG4.

IL-1β supernatant media concentrations from untreated, rhPRG4-treated, MSU treated, and MSU+rhPRG4-treated Prg4^{+/+} and Prg4^{-/-} macrophages are shown in FIG. 4C. IL-1β production was significantly higher in untreated Prg4^{-/-} macrophages compared to untreated Prg4^{+/+} macrophages (p<0.004 Similarly, IL-1β production was significantly higher in rhPRG4-treated Prg4^{-/-} macrophages compared to rhPRG4-treated Prg4^{+/+} macrophages (p<0.001). rhPRG4 treatment alone did not alter IL-1β production by Prg4^{+/+} or Prg4^{-/-} macrophages. MSU crystals significantly increased IL-1β production by Prg4^{+/+} and Prg4^{-/-} macrophages (p<0.05). IL-1β supernatant concentrations in MSU-treated Prg4^{-/-} macrophages was higher than corresponding concentrations in the MSU-treated Prg4^{+/+} macrophages (p<0.001). IL-1β supernatant concentrations in the MSU+rhPRG4 group was significantly lower than corresponding concentrations in the MSU alone group for both genotypes (p<0.004 These results indicate that PRG4 (lubricin) serve an anti-inflammatory and autocrine role in TLR-dependent inflammation from MSU crystals.

### Example 5. Impact of rhPRG4 Treatment on MSU-Induced IL-1Ra Production by Prg4^{+/+} and Prg4^{-/-} Peritoneal Macrophages

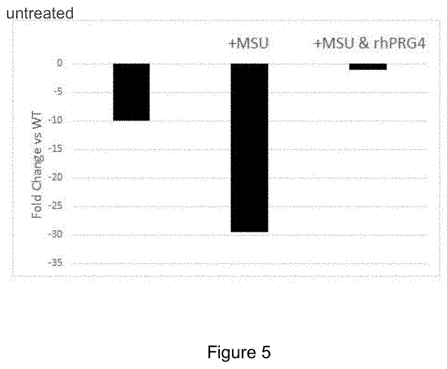

Peritoneal macrophages were harvested and pooled from 3 Prg4^{+/+} and 3 Prg4^{-/-} mice as described above. Cells were centrifuged for 10 min at 4° C. and 10,000 rpm. The supernatant was discarded and the cell pellet was gently re-suspended in RPMI 1640 media supplemented with 10% FBS and 1% penicillin/streptomycin. Cells were counted with a hemacytometer and plated onto sterile cell culture plates at a concentration of 1.3×10⁶ cells/well. Peritoneal macrophages were allowed to adhere by culturing them for 24 hours at 37° C. Treatment groups included untreated control, MSU (100 μg/mL), and MSU (100 μg/mL)+rhPRG4 (200 μg/mL). Treatments were performed for 6 hours. Following treatment, total RNA was extracted using trizol reagent (Thermo Fisher Scientific), and RNA concentrations were determined with a NanoDrop ND-2000 spectrophotometer (NanoDrop Technologies, USA). cDNA was synthesized using Transcriptor First Strand cDNA Synthesis Kit (Roche, USA). Quantitative PCR (qPCR) was performed on Applied Biosystems Step One Plus Real-Time PCR System (Thermo Fisher Scientific, USA) using TaqMan Fast Advanced Master Mix (Life Technologies, USA). Data is presented as fold change of IL-1ra expression in Prg4^{-/-} peritoneal macrophages compared with Prg4^{+/+} peritoneal macrophages under each treatment condition. Results show a reduction of IL-1ra expression in Prg4^{-/-} peritoneal macrophages compared with Prg4^{+/+} peritoneal macrophages. This reduction is exacerbated by MSU treatment, which is corrected by rhPRG4 addition (FIG. 5).

### Example 6. Crystal-Induced Mechanical Allodynia in the Rat and the Impact of rhPRG4 Treatment

Lewis rats (n=28; 10 weeks old) (Charles River, USA) were randomly assigned to two experimental groups; PBS-treated or rhPRG4-treated. All animals received an intra-articular injection of pyrogen-free MSU suspension (50 μL; 5 mg/mL). Intra-articular injections were performed under gas anesthesia (5% isoflurane). Intra-articular injections were performed in the right knee joints. The skin around the right knee joint was shaved and the injection site was cleansed using a topical iodine-based antiseptic and 70% isopropranolol. At 1 hour following MSU injection, animals received PBS (50 μL) or rhPRG4 (50 μL; 1 mg/mL).

Static weight bearing of the hind limbs of animals at baseline and at 3, 6, and 24 hours post-MSU injection was measured using an Incapacitance Meter (Harvard Apparatus, USA). Data is presented as differential weight bearing between the hind right limb and the hind left limb. At baseline, 6, and 24 hours post-MSU injections, paw withdrawal thresholds (PWT) of the hind right limb were determined using an electronic von Frey Anesthesiometer (IITC Life Sciences, USA). At each time point, PWT values were measured three consecutive times and the mean PWT of the three measurements was reported. All measurements were performed by a blinded investigator.

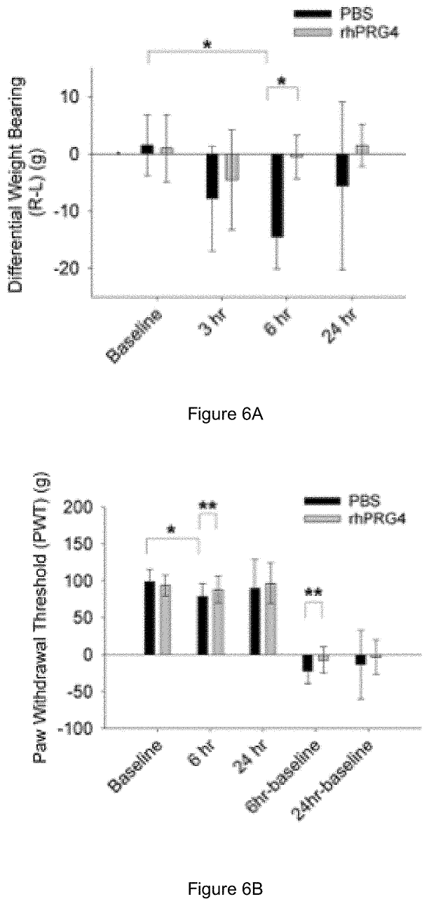

The results indicate that rhPRG4 treatment reduces mechanical allodynia following intra-articular administration of MSU crystals in the rat knee joint. Differential weight bearing of male Lewis rats at baseline and at 3, 6 and 24 hours following intra-articular administration of MSU crystals in PBS and rhPRG4-treated groups is shown in FIG. 6A. In the PBS-treated group, the differential weight bearing at 6 hours was significantly lower than the differential weight bearing at baseline (p<0.001). Additionally, the differential weight bearing in the PBS-treated group was significantly lower than the differential weight bearing in the rhPRG4-treated group at 6 hours (p<0.001). There was no significant difference in differential weight bearing between PBS and rhPRG4 treatments at 3 and 24 hours.

The PWT values at baseline and at 6 and 24 hours following MSU administration in PBS and rhPRG4-treated groups is shown in FIG. 6B. At 6 hours, the PWT values in the PBS-treated group were significantly lower than corresponding values at baseline (p<0.004 On the contrary, there was no significant difference in PWT baseline values of rhPRG4-treated animals and the corresponding values at 6 hours. The 6-hour PWT values in rhPRG4-treated animals were significantly higher than the 6-hour PWT values in PBS-treated animals (p<0.01). At 24 hours, the PWT values in the PBS-treated animals were not significantly different from corresponding baseline values or from PWT values in rhPRG4-treated animals.

### Example 7. Impact of Intravenous (IV) Administration of rhPRG4 on Rats Injected with MSU

Lewis rats of both sexes in equal proportions (n=126; 10 weeks old) (Charles River, USA) are randomly assigned to two experimental groups; PBS-treated, and rhPRG4-treated. All animals receive an IA injection of 1.25 mg MSU suspension (Invivogen). Intra-articular injection are performed in left and right knee joints as part of randomization. Intra-articular injections are performed under isoflurane vapor. After MSU injection, the knees are flexed and extended 10 times to ensure even distribution of MSU throughout the joint cavity. Both weight bearing asymmetry (Incapacitance Meter, IITC Life Sciences, USA) and paw withdrawal thresholds (PWT) are measured by an investigator blinded to which limb received MSU crystal. Static weight bearing of the hind limbs of animals at baseline and 2 hrs post MSU treatment are measured. Data are presented as differential weight bearing between the hind right limb and left limb. At the same post-MSU injection times, PWT of both hind limbs are determined using an electronic von Frey Anesthesiometer (IITC). At each time point, PWT values are measured three consecutive times and the mean PWT of the three measurements of the affected limb reported.

Two hours after the MSU IA injection, and after IITC and PWT has been measured, rats receive a single dose of IV sterile PBS (1 ml), and IV rhPRG4 (1 ml; 5.0 mg/mL). At time points of 6, 24 and 48 hrs following one of these 3 treatments, IITC and PWT are measured, and 14 animals from each are euthanized for SF analysis, synovial tissue samples obtained for histology and immunohistochemistry, flow cytometry, and serum for uric acid levels. Recovered PBMC's from the two groups of mice are analyzed for neutrophil recruitment by fluorescence-activated cell sorting using the macrophage markers F4/80, CD11b and iNOS (BD Pharmingen) SF are analyzed for PRG4 concentration, IL-1β, IL-8, MCP-1 and myeloperoxidase. Knee sections are stained with hematoxylin-eosin (H&E) to assess inflammatory cell infiltrates. The severity of cartilage damage are assessed using the OARSI Osteoarthritis Cartilage Histopathology Assessment System (OOCHAS) (OA score=grade x stage; range, 0 to 24). Statistical significance between the two groups will be assessed by two-tailed analyses. The Mann-Whitney U and two-way Analysis of variance (ANOVA) tests are computed for OOCHAS scores and continuous variables respectively. Immunohistochemistry of the synovium focuses on a) cell proliferationhistone3 and PCNA; b) CD68; c) metalloproteinases MMP-1 and MMP-13; and d) apoptosis.

Results show that rhPRG4 dosed rats exhibit decreased inflammatory arthritis assessed by mechanical allodynia, SF (synovial fluid) analysis and histology compared to rats that received PBS. Results also show that rhPRG4 administered intravenously inhibit circulating monocytes from being recruited to the gouty joint induced by intra-articular MSU administration. Results also show that PRG4 has a therapeutic effect in rats similar to the drug Anakinra, an IL-1 receptor antagonist.

### Example 8. rhPRG4 Limits the Signaling Through the TLR2 and TLR4 Receptor on Normal PMBCs by MSU Crystals

PBMCs are obtained from patients at Rhode Island Hospital (N=30) without history of gout and normal serum urate level, patients (N=30) with hyperuricemia without gout (N=30), and an acute gout flare (N=30) using MSU-crystal identification in synovial fluid as the “gold standard” for the diagnosis of gout (Schlesinger N, Rheum Dis Clin North Am 2014, 40(2):329-341; Malik A et al., J Clin Rheumatol 2009, 15(1):22-24). Patients with concomitant septic arthritis, autoimmune diseases, and other crystal-induced arthritis such as CPPD are excluded. The affecting joint is aspirated ultrasound (US) guidance and sera is collected from patients with crystal-proven acute gout during their acute gout flare.

MSU crystals (Invivogen) are suspended in sterile phosphate-buffered saline (PBS) at a stock concentration of 10 mg/ml. The morphological and birefringence properties of MSU are assessed by standard light and polarized light microscopy. The absence of microbial contaminants is evaluated for bacteria and endotoxin (<0.01 EU) by the Pyrochrome assay (Associates of Cape Cod). In sterile 96-well plates (Corning, Sigma Aldrich), 25,000 PBMCs from each normal donor (serum uric acid levels <3.0 microM) (N=30) are collected using established centrifugal Ficoll-Hypaque and Percoll gradient methods (Dagur P K et al., Current protocols in cytometry 2015, 73:5 1 1-16), split and separately plated per well using standard techniques (Crisan T O et al., Ann Rheum Dis 2016, 75(4):755-762). These PBMCs are incubated separately using normouricemic sera from the above donor patients and sera from another 30 patients with acute gout, and another 30 patients who are hyperuricemic (>3.6 microM) without gout for 24 hrs. All 90 cultured isolates are then split and treated with MSU (100 μg/mL) in the absence or co-presence of rhPRG4 (200 μg/ml) and incubated at 37° C. for 24 h. The rhPRG4, a full-length product derived from CHO-M cells (see supporting letter, Lubris, Framingham, Mass.) is utilized. Culture supernatant is assayed for IL-1β concentration and pelletized cells are processed for NLRP3/NALP3 assembly components for immunoprecipitation, IL-1ra and IL-1β protein levels and QPCR for IL-1ra expression using ACT relative to GAPDH and non-rhPRG4 treated control. In-cell Western assays ( ) are performed using the LI-COR imaging system at Tribologics (Framingham, Mass.) to confirm that the TLR receptors are occupied by the rhPRG4. In extracts from pelletized cells, immunoprecipitation experiments are performed using anti-lubricin mAb 9G3 (Ai M et al., PLoS One 2015, 10(2):e0116237) in an established immunoprecipitation protocol of PRG4 (Alquraini A et al., Arthritis Res Ther 2015, 17:353) to determine if the rhPRG4 has been cytosolically internalized, and if so, what other ligands are bound to the rhPRG4. Testing to determine if any of the components of the NLRP3/NALP3 assembly are co-immunoprecipitated with the rhPRG4 including ASC, NALP3 and TXNIP, caspase-1 and pro-IL1β is performed.

Results are expected to show that that rhPRG4 binds to the NLRP3 (NLR family, pyrin domain containing 3) inflammasome components or active caspase-1 which drives IL-1β endoproteolysis and consequent IL-1β maturation and secretion (Martinon F et al., Nature 2006, 440(7081):237-241; Martinon F et al., Mol Cell 2002, 10(2):417-426; Martinon F et al., Cell Death Differ 2007, 14(1):10-22). As a result, intravenous administration of PRG4 blocks TLR2 and TLR4 activation of monocytes to macrophages and prevents phagocytosis of MSU crystals in gouty joints.

### Example 9. Treatment of Gout in a Human Patient

A human patient suffering from an acute gouty flare up in the knee joint is administered recombinant human lubricin in an amount of 1.5 mg/kg via by intravenous administration 1-3 times in one week. After 12-48 hours the patient reports decreasing pain in the gouty joint and after 1 week, the flare up has subsided with the patient returning to normal activities and no longer complaining of pain in the joint.

### Example 10. Prevention of Gouty Flare-Up in a Human Patient

A human patient diagnosed with gout but not currently experiencing a gouty flare up is administered recombinant human lubricin in an amount of 1.5 mg/kg by intravenous administration once monthly to prevent a gouty flare up.

### Discussion of Examples

For these examples, variables were initially evaluated for normality using the Shapiro-Wilk normality test. Statistical significance comparing two groups with parametric data was assessed by Student's t test. Statistical analysis comparing multiple groups with parametric data was performed by one-way ANOVA followed by Tukey's post-hoc. Statistical significance comparing two groups with nonparametric data was assessed by Rank Sum test. Statistical significance comparing multiple groups with nonparametric data was performed by ANOVA on the ranks. All analyses were performed using Sigma Plot, version 13. A p value of <0.05 was considered statistically significant.

The forgoing examples show the activation of macrophages by MSU crystals and evaluated the consequence of rhPRG4 and macrophage interaction on MSU induced inflammation. MSU crystals induced the gene expression and production of IL-β, TNF-α, MCP-1 and IL-8 over a 24-hour period. The fold induction of gene expression and production secondary to MSU stimulation was most pronounced for IL-1β and IL-8. This observation is in agreement with previous reports demonstrating enhanced IL-1β and IL-8 expression and production in this cell model (Pazar B et al., J Immunol 2011; 186(4):2495-502; Orlowsky E W et al., BMC Musculoskeletal Disorders 2014; 15:318). rhPRG4 is shown to associate with macrophages and thereby regulates the phagocytic activity of macrophages. The down-stream effect of the rhPRG4-macrophage association is a concentration-dependent inhibition of MSU crystal phagocytosis. rhPRG4 dose-dependently reduced MSU-induced gene expression and production of IL-1β, TNF-α, MCP-1 and IL-8. TNF-α and IL-8 gene expression and production were most susceptible to the inhibitory effect of rhPRG4. Overall, rhPRG4 exhibited an anti-inflammatory activity at physiologically relevant concentrations that have been previously reported in SF aspirates from normal subjects and from patients with OA (Kosinska M K et al., PLoS One 2015; 10:e0125192).

PRG4 plays a homeostatic role in the articular joint with an established role in regulating synovial overgrowth and preserving cartilage integrity (Jay G D et al., Matrix Biol 2014; 39:17-24; Rhee D K et al., J Clin Invest 2005; 115(3):622-31). Histological features in joints from Prg4 knockout mice include synovial hyperplasia, cartilage surface fibrillations and chondrocyte apoptosis (Rhee D K et al., J Clin Invest 2005; 115(3):622-31; Jay G D et al., Arthritis Rheum 2007; 56(11):3662-9; Waller K A et al., Proc Natl Acad Sci USA 2013; 110(15):5852-7). These pathological changes appear irreversible even with restoration of Prg4 expression (Hill A et al., Arthritis Rheumatol 2015; 67(11):3070-81). Interestingly, synoviocytes isolated from knee synovial tissues of Prg4 knockout mice exhibit a pro-inflammatory phenotype characterized by enhanced basal and cytokine induced proliferation compared to synoviocytes isolated from wild type animals (Al-Sharif A et al., Arthritis Rheumatol 2015; 67(6):1503-1513). As described herein, isolated peritoneal macrophages from Prg4 knockout and wild type animals were studied along with their MSU phagocytic activity as well as their subsequent activation. A 6 fold enhanced MSU uptake by Prg4 knockout macrophages compared to wild type macrophages was observed. The uptake of MSU by Prg4 knockout and wild type macrophages was mediated by TLR2 and rhPRG4 was efficacious in suppressing MSU uptake by macrophages from both genotypes. An enhanced basal production of IL-1β by Prg4 knockout macrophages with ˜2-fold enhancement compared to wild type macrophages was also observed. Interestingly, basal IL-1β production was not influenced by rhPRG4 treatment. Upon MSU phagocytosis, IL-1β production by Prg4 knockout macrophages remained higher than IL-1β production by wild type macrophages. MSU challenge resulted in a disproportionate increase (˜14-fold) in IL-1β production by Prg4 knockout macrophages in relation to wild type macrophages. These findings support that PRG4 may have a biological role in regulating the activation of tissue macrophages by TLR ligands, notably MSU crystals.

Phagocytosis of MSU crystals by human and murine macrophages and IL-1β production was reversed by TLR2 neutralization. This observation supports a role for TLR2 in mediating the initial steps of gout pathogenesis (Bryan R L et al., Arthritis Rheum 2005; 52(9):2936-2946; Joosten L et al., Arthritis Rheum 2010; 62(11):3237-3248). While TLR2 is important in mediating the phagocytosis of MSU crystals, it may not play a significant role in sustaining the inflammatory response and neutrophil influx in vivo (Chen C J et al., J Clin Invest 2006; 116:2262-2271). The mechanism of inhibition of MSU phagocytosis by rhPRG4 is probably related to its ability to bind to TLR2 receptors on the surface of macrophages. However, the contribution by other, yet to be identified, surface receptors cannot be ruled out. PRG4 was shown to bind to other cell surface receptors, e.g. CD44 (Al-Sharif A et al., Arthritis Rheumatol 2015; 67(6):1503-1513). The complexity of the interaction between PRG4 and cell surfaces is further highlighted by the unique structure of PRG4. PRG4's protein core is 1,404 amino acid long with N and C termini and a central mucin domain that is heavily glycosylated via O-linked β(1-3)Gal-GalNAc oligosaccharides, allowing it to assume a unique brush-like polymeric conformation (Zappone B et al., Biophys J 2007; 92(5):1693-1708). PRG4 was previously shown to bind to L-selectin in a glycosylation-dependent manner (Estrella R P et al., Biochem J 2010; 429(2):359-67; Jin C et al., J Biol Chem 2012; 287(43):35922-33). Additionally, PRG4 amino terminal domains are homologous to somatomedin B domain of vitronectin and the carboxy terminal contains a hemopexin domain and may mediate surface binding of the protein (Jones A R et al., J Orthop Res 2007; 25(3):283-92).

IL-1β plays a pivotal role in mediating gouty inflammation and IL-1 inhibitors were shown to relieve pain and inflammation in rodent models and in clinical experience (Tones R et al., Ann Rheum Dis 2009; 68(10):1602-8; Edwards N L et al., Rheum Dis Clin North Am. 2014; 40(2):375-87; Ottaviani S et al., Arthritis Res Ther 2013; 15(5):R123). IL-1 inhibitors do not interfere with MSU phagocytosis by macrophages and other cells in the joint, and the resultant expression and production of pro-inflammatory cytokines and chemokines. IL-1 inhibitors block the autocrine and paracrine effects of locally produced IL-1β and hence the downstream inflammatory cascade. rhPRG4 works at an earlier point in the gout inflammatory pathway by reducing MSU phagocytosis. The mechanism of action of rhPRG4 results in an indirect IL-1 antagonist effect, via reducing IL-1β production and hence attenuating its role in driving gout pathogenesis.

Intra-articular administration of MSU resulted in an acute mechanical allodynia that peaked at 6 hours following MSU administration and gradually resolved by 24 hours. This timeline is in accordance with previous reports that demonstrated that synovial tissue COX2 gene expression and associated mechanical allodynia are significantly increased following MSU administration in rat knee or ankle joints (Coderre T J et al., Pain 1987; 28:379-393; Lee H S et al., Osteoarthritis Cartilage 2009; 17:91-99; Silva C R et al., Ann Rheum Dis 2016; 75(1):260-8). rhPRG4 treatment reduced mechanical allodynia and normalized animals' weight bearing. This novel in vivo anti-nociceptive and anti-inflammatory efficacy of rhPRG4 builds upon previous reported efficacy of rhPRG4 in pre-clinical posttraumatic osteoarthritis models (Jay G D et al., Arthritis Rheum 2012; 64(4):1162-71; Jay G D et al., Arthritis Rheum 2010; 62(8):2382-91; Cui Z et al., Bone 2015; 74:37-47; Teeple E et al. Am J Sports Med 2011; 39(1):164-72) and provides a rationale for further investigation of rhPRG4's efficacy as a treatment for acute gout by preventing MSU phagocytosis.

PRG4 is a glycoprotein with a multifaceted role in the articular joint. By virtue of association with macrophage surface receptors e.g. TLR2, rhPRG4 functions to inhibit MSU crystal phagocytosis by human and murine macrophages and resultant induction of gene expression and production of key inflammatory mediators e.g. IL-1β and key chemotactic cytokines e.g. IL-8. All these effects are therapeutically beneficial in acute gout flares. rhPRG4 efficacy extends to an in vivo acute gout model, where rhPRG4 treatment reduced mechanical allodynia and normalized weight bearing.

1. The method of claim 1, wherein the method reduces joint pain in the subject.

2. A method of treating gout or pseudogout in a subject, the method comprising administering to a subject by systemic administration a composition comprising PRG4 or a biologically active fragment thereof.

3. The method of claim 1, wherein the method decreases phagocytosis of monosodium urate monohydrate (MSU) crystals by a macrophage in the subject.

4. The method of claim 1, wherein the method reduces inflammation associated with gout in the subject.

5. The method of claim 2, wherein the PRG4 is recombinant human PRG4.

6. (canceled)

7. (canceled)

8. The method of claim 2, wherein the systemic administration is intravenous administration.

9. (canceled)

10. (canceled)

11. The method of claim 2, wherein the composition further comprises a pharmaceutical carrier.

12. The method of claim 2, wherein the PRG4 is administered in an amount insufficient to provide boundary lubrication but sufficient to treat joint pain or allodynia.

13. The method of claim 2, wherein the PRG4 is administered in an amount in the range of 0.05 mg/kg to 1.50 mg/kg, 0.1 mg/kg to 100 mg/kg, 0.5 mg/kg to 50 mg/kg, 1 mg/kg to 100 mg/kg, 1 mg/kg to 25 mg/kg, 1 mg/kg to 10 mg/kg, 2 mg/kg to 10 mg/kg, or 5 mg/kg to 10 mg/kg.

14. The method of claim 2, wherein the PRG4 is administered in an amount in the range of 0.1 μg/mL to 30 mg/mL, 1 μg/mL to 10 mg/mL, or 10 μg/mL to 1 mg/mL.

15. The method of claim 2, wherein the PRG4 is administered in an amount sufficient to achieve a concentration of PRG4 in a synovial fluid of a joint of the subject of at least 200 μg/ml, at least 300 μg/ml, at least 400 μg/ml, at least 500 μg/ml, or at least 1000 μg/ml.

16. The method of claim 2, wherein the PRG4 is administered in the range of 2 mg to 10 mg, 2 mg to 5 mg, or 5 mg to 10 mg.

17. The method of claim 2, wherein the PRG4 is administered in an amount greater than 10 mg.

18. The method of claim 2, wherein the subject is a mammal.

19. The method of claim 18, wherein the subject is a human, horse, sheep, pig, dog, or cat.

20. The method of claim 2, wherein the PRG4 is administered weekly, biweekly, monthly or quarterly.

21. (canceled)

22. (canceled)

23. (canceled)

24. (canceled)

25. (canceled)

26. (canceled)

27. The method or use of claim 2, wherein the PRG4 or biologically active fragment thereof has at least 80%, at least 85%, at least 90%, at least 95%, at least 96%, at least 97%, at least 98%, or at least 99% sequence identity with SEQ ID NO: 1.

28. The method of claim 2, wherein the systemic administration is intramuscular administration, oral administration, subcutaneous administration, intradermal administration, transmucosal administration, nasal administration, inhalational administration, rectal administration, sublingual administration, sublabial administration, or buccal administration.
